(19) United States
(12) Patent Application Publication
(10) Pub. No.: US 2016/0122794 A1
(43) Pub. Date: May 5, 2016

(54) SYSTEM, METHOD AND APPARATUS FOR PATHOGEN DETECTION

(71) Applicant: SIGHTLINE INNOVATION INC., Toronto (CA)

(72) Inventors: Wallace TRENHOLM, Toronto (CA); Jason CASSIDY, Hamilton (CA)

(21) Appl. No.: 14/928,313

(22) Filed: Oct. 30, 2015

Related U.S. Application Data
(60) Provisional application No. 62/157,041, filed on May 5, 2015.
(60) Provisional application No. 62/081,152, filed on Nov. 18, 2014.
(60) Provisional application No. 62/072,590, filed on Oct. 30, 2014.

(51) Int. Cl. C12Q 1/04; G01N 21/45; G01N 21/65

(52) U.S. Cl. CPC C12Q 1/04; G01N 21/658; G01N 21/45; G01N 2201/06113

(57) ABSTRACT

Systems and methods for pathogen detection are described. A method for pathogen detection comprises collecting a sample from a subject, combining the sample with amplification substances, performing detection operations on the combined sample, analyzing the detection signals, and disposing of the combined sample. Embodiments of an apparatus for pathogen detection comprise a microfluidic disk having a plurality of reaction chambers for combining a sample and amplification substances. Further described apparatuses for pathogen detection comprise a storage unit, a sensor unit and a disposal unit.

### TECHNICAL FIELD

The following relates generally to pathogen detection and more specifically for systems, method and apparatus for detection of a pathogen from a sample.

### BACKGROUND

Today, as long distance travel is becoming readily available, quick detection of pathogens such as the Ebola virus is becoming vital in the fight against the spread of infections. Specifically, as people travel by fast public transport such as planes and trains, availability of rapid detection mechanisms for screening passengers, pets and other live animals for the presence of infectious pathogens is increasingly of vital importance.

Pathogen detection systems presently available are highly invasive and slow. For example, a typical method for the detection of an Ebola infection involves withdrawing a blood sample and sending the sample to a lab for analysis, which typically takes one or more days. Accordingly, a system and method is needed for rapid detection of pathogens.

These and other aspects are contemplated and described herein. It will be appreciated that the foregoing summary sets out representative aspects of systems, methods, apparatus for pathogen detection to assist skilled readers in understanding the following detailed description

### SUMMARY

These and other aspects are contemplated and described herein. It will be appreciated that the foregoing summary sets out representative aspects of systems, methods, apparatuses for in pathogen detection to assist skilled readers in understanding the following detailed description.

In one aspect, a system for detecting a pathogen is provided, the system comprising: a collector for collecting a sample from a subject; an assembly for receiving the sample, the assembly comprising: a substrate layer; and an amplification layer comprising at least one amplification substance immobilized and functionalized to the substrate layer for interacting with a desired substance associated with the presence of the pathogen in the sample; a detector for receiving the assembly and for generating detection signals from the received assembly according to at least one detection modality; and a computing device for analyzing the detection signals and for determining presence or absence of the pathogen in the sample.

In another aspect, a method for detecting a pathogen is provided, the method comprising: receiving a sample from a subject using a collector; providing the sample to an assembly, the assembly comprising: a substrate layer; and an amplification layer comprising at least one amplification substance immobilized and functionalized to the substrate layer for interacting with a desired substance associated with the presence of the pathogen in the sample; providing the assembly to a detector, the detector configured to generate detection signals corresponding to the assembly according to at least one detection modality; and initiating the determination, by a computing device having a processor, of the presence or absence of the pathogen in the sample by analyzing the detection signals.

In another aspect, an apparatus for detecting a pathogen is provided, the apparatus comprising: a collector storage vessel for storing at least one collector for collecting a sample from a subject; a storage unit for storing a plurality of assemblies, each assembly comprising at least one amplification substance for interacting with a desired substance associated with the presence of a pathogen; a detector unit for generating detection signals from a selected assembly from the plurality of assemblies according to at least one detection modality; a computing device for analyzing the detection signals and determining the presence or absence of the pathogen in the sample; a disposal unit for decontaminating the selected assembly; and a sample handling unit comprising a robotics controller and mechanical linkages for: receiving a collector from the subject; retrieving the selected assembly from the storage unit; combining the sample and the selected assembly; and providing the combined sample and selected assembly to the detector unit, and the disposal unit.

In another aspect, a method for detecting a pathogen is provided, the method comprising: providing to an apparatus a collector having collected a sample from a subject, the apparatus configured to: store a plurality of assemblies, each assembly comprising at least one amplification substance for interacting with a desired substance associated with the presence of a pathogen; retrieve, a selected assembly from the plurality of assemblies; combine, the sample and the selected assembly; generate detection signals, by a detector unit, from the combined assembly according to at least one detection modality; analyze, by a computing device, the detection signals and determine the presence or absence of the pathogen in the sample; and decontaminate the combined assembly.

In another aspect, an apparatus for detecting a pathogen is provided, the apparatus comprising a microfluidic disk comprising a top layer and a bottom layer disposed in a mating relationship along a mating surface, the bottom layer comprising a substrate layer, the top layer comprising: a sample port formed by an aperture disposed through the center thereof; one or more microchannels formed along the mating surface, each of the microchannels extending radially from the sample port toward the periphery of the microfluidic disk; and one or more reaction chambers formed along the mating surface, each of the reaction chambers disposed part way along a respective one of the microchannels

In another aspect, a method for pathogen detection comprising; receiving a sample from a collector; providing the sample to a sample port of a microfluidic disk comprising a top layer and a bottom layer disposed in a mating relationship along a mating surface, the bottom layer comprising a substrate layer, the top layer comprising: a sample port formed by an aperture disposed through the center thereof; one or more microchannels formed along the mating surface, each of the microchannels extending radially from the sample port toward the periphery of the microfluidic disk; and one or more reaction chambers formed along the mating surface, each of the reaction chambers disposed part way along a respective one of the microchannels centrifuging the microfluidic disk by a centrifugation unit; receiving the microfluidic disk at a detector; generating detection signals by the detector from at least one of the one or more reaction chambers according to at least one detection modality; and analyzing the detection signals by a computing device for generating a determination of the presence or absence of the pathogen in the sample.

### BRIEF DESCRIPTION OF THE DRAWINGS

A greater understanding of the embodiments will be had with reference to the Figures, in which:

FIG. 1 shows a system for the detection of a pathogen in accordance with an implementation;

FIG. 2 shows a method of pathogen detection using the system of FIG. 1 in accordance with an implementation;

FIG. 3 shows a cross section of an assembly for combining a sample with amplification and enhancer substances in accordance with an implementation;

FIG. 4 shows a collector with a rupturable buffer chamber;

FIG. 5 shows a microfluidic disk comprising a plurality of reaction chambers;

FIG. 6 shows embodiments of microfluidic disks with different sets of amplification substances in reaction chambers;

FIG. 7 shows a pathogen binding to an amplification substance in an assembly of a reaction chamber;

FIG. 8 shows a centrifugation unit for monitoring the spread of a sample throughout the microfluidic disk;

FIG. 9 shows an example embodiment of a microfluidic disk comprising a Bragg grating;

FIG. 10 shows performing surface plasmon resonance on the assembly of FIG. 3 in accordance with an implementation;

FIG. 11 shows performing backscattering interferometry on the assembly of FIG. 3 in accordance with an implementation;

FIG. 12 shows an apparatus for pathogen detection;

FIG. 13 shows a sensing module of the apparatus of FIG. 12;

FIG. 14 shows a flowchart of a method of pathogen detection using the sensing module of FIG. 13;

FIG. 15 shows a detection operation performed on a microfluidic disk; and

FIG. 16 shows another apparatus for pathogen detection and a flowchart of a method of pathogen detection using the apparatus;

FIG. 17 shows another embodiment of a microfluidic disk; and

FIG. 18 shows yet another embodiment of a microfluidic disk;

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical discs, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile discs (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

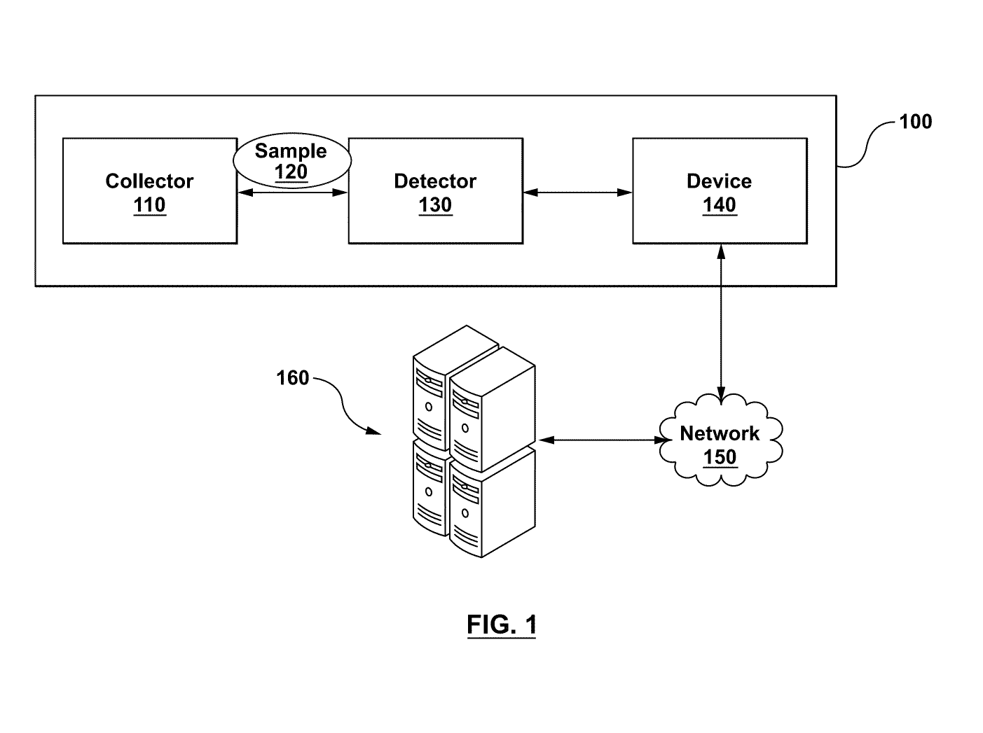

A system for pathogen detection is shown, in accordance with an implementation, generally at 100 in FIG. 1. As described in more detail below, the system 100 comprises a collector 110 for collecting a sample 120, a detector 130 for detecting the presence of specific substances in the sample and a computing device 140 for performing an analysis of detection signals received from the detector 130. The device 140 is operable to connect to one or more remote computers 160 through the network 150, which can be a personal, local or wide area network, such as the Internet. In variations, the device 140 may not be part of the system 100. In such variations, the detector 130 can be operable to connect to the remote computers 160 through the network 150, the remote computers 160 performing the functionality of the device 140. In some implementations, the system 100 is a mobile system being transportable to different locations, for example, for use in field operations. In variations, the system can be fixed, such as a system for use at airport security locations.

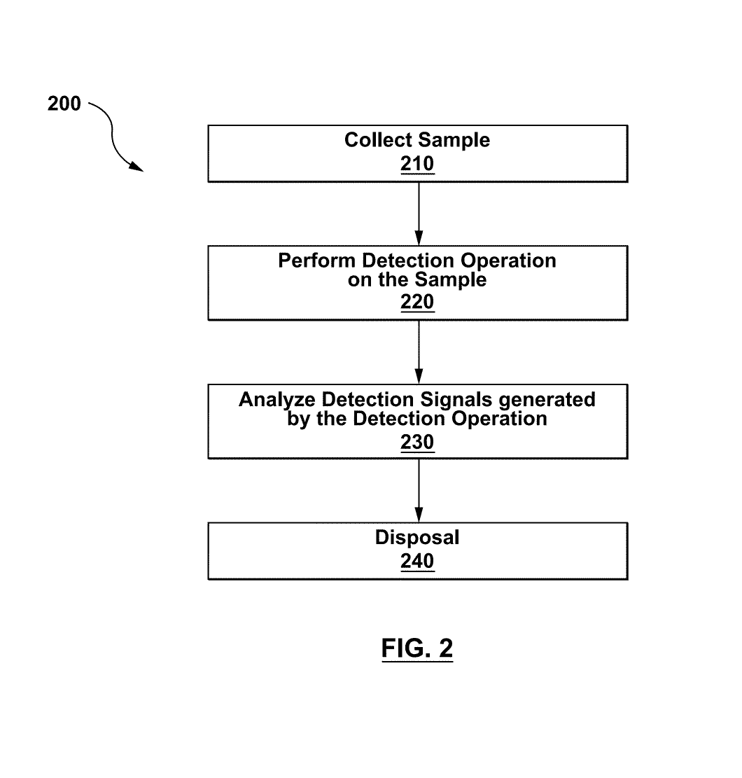

Referring to FIG. 2, a method of pathogen detection is indicated at 200. At block 210, the sample is collected by the collector 110. The collector 110 can take various forms suitable for the collection of a desired form of the sample 120. Once collected, the sample may be processed. At block 220, detection operations are performed on the sample by a detector 130 to generate detection signals. At block 230, detection signals are analyzed by computing device 140 or remote computers 160 for detecting the presence of a desired substance. At block 240, the sample is disposed of.

The blocks of method 200 will now be described in additional detail.

Referring now to block 210, a desired sample is a sample 120 which contains concentrations of a desired substance to be detected. The desired substance to be detected can be the pathogens to be detected or indicators of the presence of the pathogens to be detected.

For example, in some implementations, the sample provider can be a person and the pathogen to be detected can be the Ebola virus (EBOV). One indicator of infection by the Ebola virus is an antigen related to the EBOV such as a glycoprotein related to the EBOV. For example, infection by the EBOV can cause the transcriptional editing of the fourth gene (GP) resulting in the expression of a 676-residue transmembrane-linked glycoprotein termed GP, as well as a 364-residue secreted glycoprotein termed sGP. The EBOV and thus the GP and the sGP expressed can vary based on various species of the EBOV. For example, the glycoproteins can be related to the Zaire Ebola virus (ZEBOV GP), which is the desired substance in the present example (hereinafter the glycoprotein). In variations, glycoproteins related to different species of Ebola filoviruses similar to the ZEBOV such as the Sudan Ebola Virus (SEBOV) GP, Bundibugyo Ebola Virus (BEBOV) GP, Reston Ebola Virus (REBOV) GP, Lassa virus GP, and Marburg virus GP can be the desired substance. The expressed glycoprotein can be found in blood and other various bodily fluids, such as a person's saliva, semen, breast milk and other mucosal secretions. Accordingly, in some implementations, such as the present illustrative example where the ZEBOV is the pathogen being detected and the ZEBOV GP is the desired substance, the sample 120 can be a bodily fluid such as saliva.

Though the teachings below, in some instances, describe glycoproteins associated with ZEBOV GP as a desired substance, these examples are merely illustrative. Various pathogens and associated desired substances are contemplated with necessary modifications and alterations to the described examples, such as selecting appropriate amplification substances for detecting the desired substances (as described below). The desired substance may relate to other hemorrhagic fevers, such as Lassa and Marburg. The desired substance may relate to bioterror pathogens, such as Anthrax, Smallpox or the Plague. The desired substance may relate to environmental pathogens, such as Escherichia coli, Cryptosporidium or salmonella. The desired substance may relate to sexually transmitted infections, such as human immunodeficiency virus, Herpes simplex virus or Treponema pallidum. The desired substance may relate to oncology biomarkers, such as CA-125, BRCA1/BRCA2 or CA 19-9. Further, though the term “pathogen” is used herein, the systems and methods are applicable to the detection of other types of substances and materials with necessary modifications that will be apparent to those of skill in the art.

In implementations where the sample 120 is saliva, the collector 110 can be in a form suitable for collecting saliva, such as a container into which the sample provider can spit or a swab which can be used to swab the inside of the mouth or the nasal passages.

Once the sample 120, in an illustrative example saliva, is collected by the collector 110, the desired substance can be detected by performing one or more detection operations on the sample 120 at block 220. In some implementations, the sample 120 can be processed to amplify or enable the detectability of the desired substance. For example, in some implementations, one or more amplification substances can be combined with the sample 120. The amplification substances can be substances designed to bind to, or otherwise interact with, the desired substance, amplifying its detectability as a result of the binding. Accordingly, in the present example, an amplification substance can be antibodies, aptamers, or molecular imprinted polymers specific to the glycoproteins or specifically to ZEBOV GP. Further, the amplification substances may be monoclonal antibodies, Fab fragments, single-domain antibodies, single-chain fragment variable (scFv), and molecular imprinted polymers, DNA, RNA. It will thus be appreciated that the amplification substances generally provide assays, such as bioreceptors, for facilitating detection of particular desired substances.

In some implementations, the amplification substances can include one or more enhancer substances. The enhancer substances can further increase or further enable the detectability of the amplification substances. For example, the antibodies of the present example can be bound to enhancer particles such as conductive particles including metal particles such as gold, copper and/or silver nanoparticles. The metal nanoparticles can typically further enhance the detectability of the antibodies. For example, the metal nanoparticles can act to amplify the signals produced by a source laser of a Raman spectrometer by manipulating the end of the antibodies. In variations described in more detail below, the metal nanoparticles can include a passive layer such as a silica coating. The amplification substances can be in the form of solutions, substrates, prefabricated test materials and others which will now occur to a person of skill. In the present example, the amplification substance is the glycoprotein antibody KZ52. The KZ52 antibody was derived originally from a patient who survived a Zaire Ebola outbreak in the 1990s. The enhancer substance is a 30 nm gold nanoparticle, that is thinly silica coated (1 nm thickness) and conjugated to anti-Zaire Ebola glycoprotein antibody KZ52. The particle size can vary from 10 nm to 50 nm. The coating thickness can vary from 0.5 nm to 3 nm. The antibody KZ52 can be, for example, in the form of a human anti-ZEBOV-GP functionalized IgG solution (2.5 mg/ml).

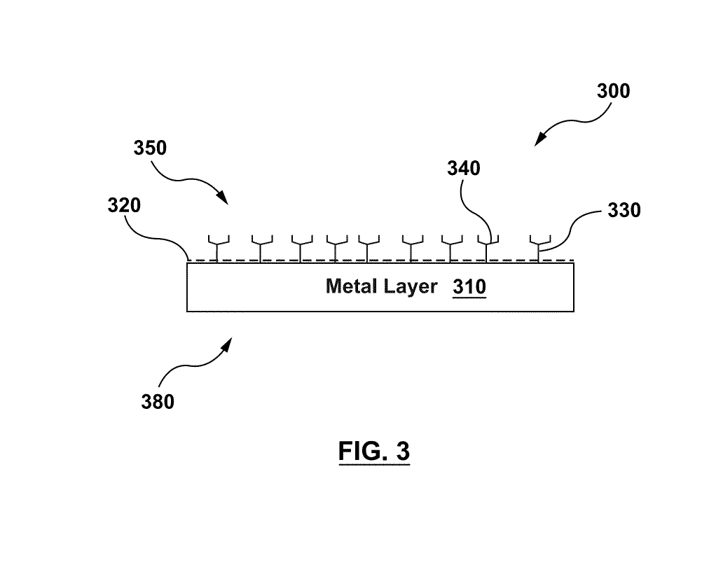

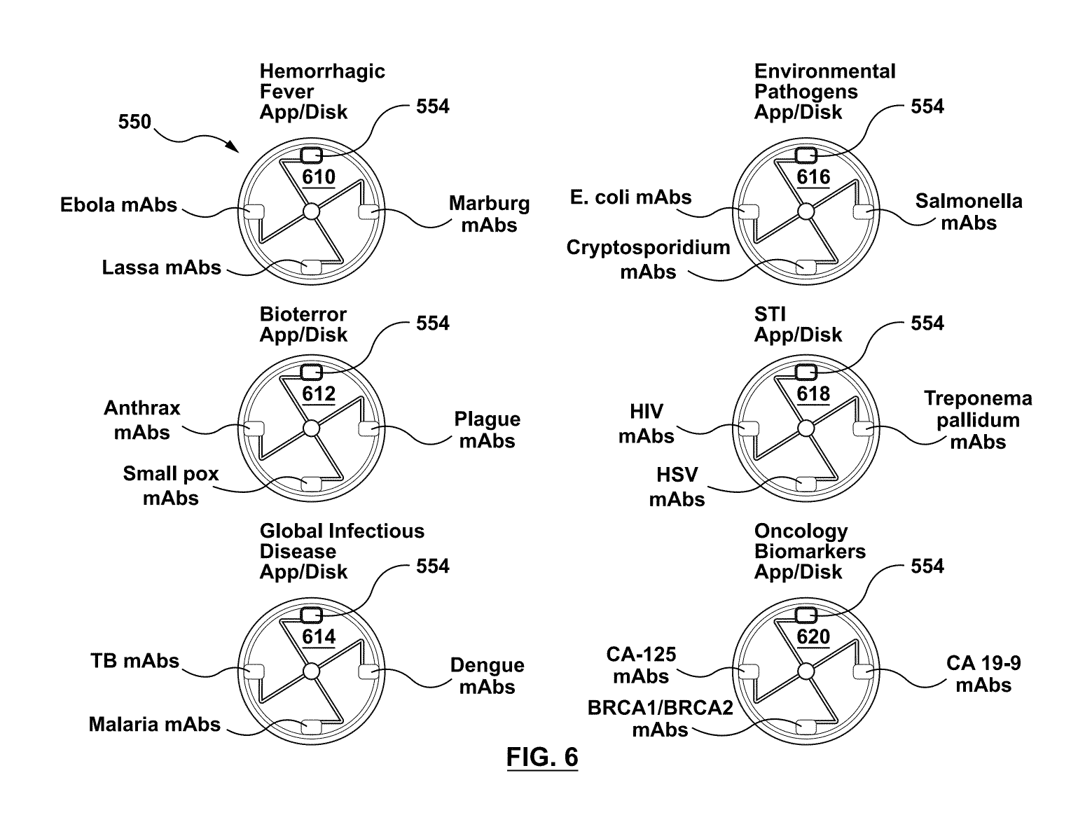

FIG. 3 shows a cross section of an assembly for use in combining a sample with an amplification substance and an enhancer substance. In accordance with this non-limiting example, the enhancer substance can be a conductive layer such as a metal layer 310. The metal layer 310 can be formed from gold, silver or copper, or nanoparticles thereof, for example, and can have a thickness at a nanometer scale. The metal layer 310 can be coated with an insulating layer 320, shown as a dotted line in FIG. 3. The insulating layer 320 can be formed from silica, glass, SiO₂/Si, quartz or other materials such as glycol-derivate oligo/polymers. It is noted that the ratio of the thicknesses of the insulating layer 320 and the metal layer 310 shown in FIG. 3 are chosen purely for illustrative purposes and do not indicate any limitations on the thickness ratios for the two layers. The amplification substance, in this example being the antibodies discussed above and shown at 330, can be attached to the insulating layer 320 or the metal layer 310. As illustrated in FIG. 6, other amplification substances are also contemplated, depending on the target pathogen. In accordance with the example implementation of FIG. 3, the attached antibodies 330 can be immobilized to the insulating layer 320 by electrostatic interactions, covalent coupling (coupling of nucleophiles to carboxylic groups, thiol groups or aldehyde groups) and coupling of native and tagged molecules (avidin/biotin). The amplification substances may further be attached to the insulating layer or the metal layer by noncontact biodeposition, such as piezoelectric inkjet printing or laser direct writing. Other processes for attaching the attached antibodies 330 to the metal layer 310 or the insulating layer 320 will now occur to a person of skill and are contemplated and described in more detail below. The attachment is performed in such a manner that the portions of the amplification substance to which the desired substance, such as the glycoprotein in this example, bind or interact with, remain exposed, as indicated at 340. The density of the attached antibodies 330 on the surface of the layers 310 and/or 320 as well as the uniformity of the distribution of the attached antibodies 330 on the surface of the layers 310 and/or 320 can vary. Each liquid amplification substance (such as antibody) “drop” may have a concentration of ˜0.5 mg/mL, and may measure approximately ˜100 um in diameter.

Amplification substances, and more specifically the biomolecules thereof (such as antibodies), may thus be attached to the top surface 350 of assembly 300. The surface may be prepared as described in Advances in Plasmonic Technologies for Point of Care Applications, Onur Tokel, Fatih Inci, Utkan Demirci, Chem Rev. 2014 Jun. 11; 114(11): 5728-5752. Chemical adsorption and covalent binding techniques may provide chemical coupling and bond formation between surface and biomolecules in multiple steps including support surface activation, functional group generation, and biomolecule immobilization. A particular chemical adsorption technique called the self-assembled monolayer (SAM) technique spontaneously generates selfformation of molecular assemblies on substrates. Nalkylthiols or disulfides are the most common SAM molecules. Typical biomolecule immobilization methods include coupling reactions (e.g., Nhydroxysuccinimide (NHS) and ethyl(dimethylaminopropyl)carbodiimide (EDC)) that may form succinimide groups that interact with amine groups of organic molecules (e.g., antibody, protein, nucleic acids, and aminemodified lipids). SAMs can also be utilized to block the surface from nonspecific binding. Avidin-biotin based interactions may be used to immobilize biomolecules on the biosensing surface (i.e. the surface 350 of the assembly 300). Protein G has a specific binding site for the fragment crystallizable region (Fc) of antibodies, such that it provides good control over antibody orientation. Immunoglobin specific proteins can be engineered using recombinant DNA technology to increase the number of binding sites and to increase stability. It is understood that nonspecific binding to biosensing surfaces is a drawback of current biosensing methods, given that the concentration of other substances (i.e. undesired, non-specific substances) may be higher than the target analyte and that these substances can also bind/attach to the biosensing area, though providing different binding characteristics. Further, nonspecific binding can occur at functionalized, passivated, and untreated regions of the biosensing area. Thus, these nonspecific interactions can decrease detection sensitivity. Antifouling agents (e.g., chemical, protein based, and polymeric agents) may be used to address these challenges by improving the specificity. Thiol compounds may be used as chemical blocking agents on metal surfaces. Proteins (e.g., bovine serum albumin, casein, glycine, and gelatin) may also been used to protect the biosensing surface from nonspecific interactions. However, these natural blocking agents (e.g., albumin, casein, and glycine) may not be satisfactory. Polymeric blocking agents are easily reproducible and can be modified to increase the specificity of blocking. The combination of long and short PEG chains significantly reduces biofouling on the biosensing surface and increases the sensitivity, such as a copolymer (i.e., poly(ethylene glycol)bpoly(acrylic acid) (PEGbPAAc)). The sensitivity may be further improved by using dual polymers (i.e., PEGbPAAc and pentaethylenehexamineterminated PEG (N6PEG)). The use of dual polymers may be found to demonstrate higher sensitivity and reliability for the biosensing surfaces where a small amount of target molecules from complex fluids such as whole blood is detected. Further, in addition to the above surface modifications, the generation of nanorough surfaces may minimize bacterial attachment on biosensing surface.

The combining of the amplification substances with the sample can be accomplished using various methods. For example, a swabbed sample 120 can be dipped into a solution and/or rubbed onto a substrate or a test material containing the amplification substances. In variations, such as the present example where the sample 120 is a liquid collected in container, the amplification substances (in powder form or in a solution, for example) can be added to the sample 120. In case of the example of FIG. 3, to combine the amplification substances with the desired substance, the sample 120 is applied to the top surface 350 of the assembly 300. In some implementations, the collection of the sample 120 and the combination of the amplification substances and the desired substance can be done simultaneously by making the assembly 300 part of a swab. Accordingly, the top surface 350 can be rubbed on a sample 120, such as a person's cheek, as part of using the swab.

In some variations, once the amplification substances are combined with the sample 120 (hereinafter the combined sample), an incubation period can elapse prior to performing a detection. The incubation period can allow the proper combination of the desired substance with the amplification substances, for example allowing them to appropriately bind. The incubation period can be in the order of hours, minutes or seconds, based on the specific substances and the detection methods used.

In further variations, the sample 120 can be additionally processed, chemically and/or mechanically, prior to or after combination with the amplification substances. For instance, in the present example where the saliva is collected in the collector 110, the sample 120 can be centrifuged and the resulting supernatant removed prior to combining with the amplification substances. In one variation, the concentration of the desired substance in the sample 120 can be adjusted based on processing to bring it to a level between 1000 parts per million (ppm) and 1 ppm.

In some variations, once the amplification substances are combined with the sample 120, and optionally after an incubation period, a buffer or cleaning solution may be discharged from a buffer solution storage chamber (such as the rupturable chamber 418 described below) by a buffer application unit to the combined sample in order to flush off or reduce the concentration of any remaining sample substances that have not bound (i.e. non-specific substance) to the amplification substances. In case of the example of FIG. 3, once the desired substance has attached to the portions of the amplification substance to which the desired substance binds or interacts 340, a buffer solution may be applied to the assembly 300. After the buffer solution is applied, or contemporaneously therewith, the buffer solution may be removed. Various embodiments of the buffer solution are contemplated. In some embodiments the buffer solution may be a phosphate buffered saline solution (“PBS”), or may be TrisT+, TrisT−, Bovine serum albumin (BSA”), and/or PBS. Application of the buffer solution may improve accuracy of detection operations 220 by reducing noise level, such as by reducing noise from any remaining non-bound sample substances and amplification substance, and optionally by reducing temperature fluctuations. In alternate embodiments, detection operations 220 can be performed while the buffer solution remains. In such embodiments, the characteristics of the buffer solution will be selected to facilitate detection operations 220. For example, a buffer solution having appropriate optical characteristics, such as refractive index, for the particular selected detection modality of the detection operations 220 will be selected.

The sample 120 collected by the collector 110 can thus be combined with amplification substances (such as on assembly 300) and optionally a buffer solution prior to carrying out detection operations.

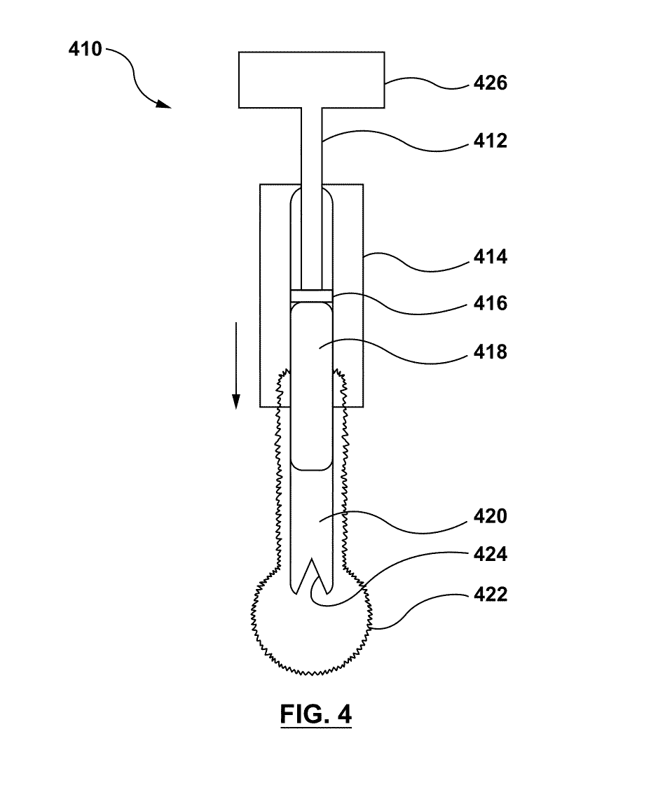

Referring now to FIG. 4, an embodiment of collector 110 for collecting a sample 120 and for facilitating application of a buffer solution is shown. The collector 410 has a buffer solution storage chamber 418 (which may be external to or integral with the collector) for dispensing a buffer solution to the combined sample; accordingly the collector 410 is an example embodiment of a buffer application unit. Preferably, application of the sample 120 to amplification substances precedes application of the buffer solution.

More particularly, FIG. 4 shows an illustrative embodiment of a collector 410 comprising a plunger 412, a barrel 414, a rupturable buffer chamber 418 housing a buffer solution, a chamber 420 having a piercing element 424, and an absorbant fluid collector 422. The rupturable buffer chamber 418, chamber 420 and absorbant fluid collector 422 are shown to be retained at least partly within the barrel 414.

The absorbant body fluid collector 422 may have the characteristics of a swab. When the absorbant body fluid collector 422 is placed into contact with a sample fluid, the sample fluid is absorbed therein. In embodiments, the components are mechanically linked such that retraction of the plunger 412 reduces the pressure in the chamber 420 causing sample fluid in proximity to the collector 422 to be sucked into the chamber 420. The plunger 412 comprises a handle 426 external to the barrel for depressing the plunger and a plunger foot portion 416 for pushing the rupturable buffer chamber 418 onto a piercing element 424. Upon depression of the plunger 412, the rupturable buffer chamber 418 is pressed against the piercing element 424, causing the rupturable buffer chamber 418 to rupture and dispense buffer solution. Partial depression of the plunger may discharge the sample without rupturing the buffer chamber 418.

In use, the collector 410 will be applied to a subject to collect a sample, such as by swabbing the absorbant body fluid collector 422 against a subject's cheek. In some embodiments, the plunger may be retracted to enhance sample collection. Once the sample 120 is collected at the absorbant collector 422, the sample 120 may be combined with amplification substances. For use with assembly 300, to deposit the sample, the collector 410 may be swabbed against the assembly 300, and the plunger may partially depressed in order to expel the sample without rupturing the rupturable buffer chamber. Subsequently, or contemporaneously, the plunger can be fully depressed to pierce the rupturable buffer chamber 418 and release the buffer solution.

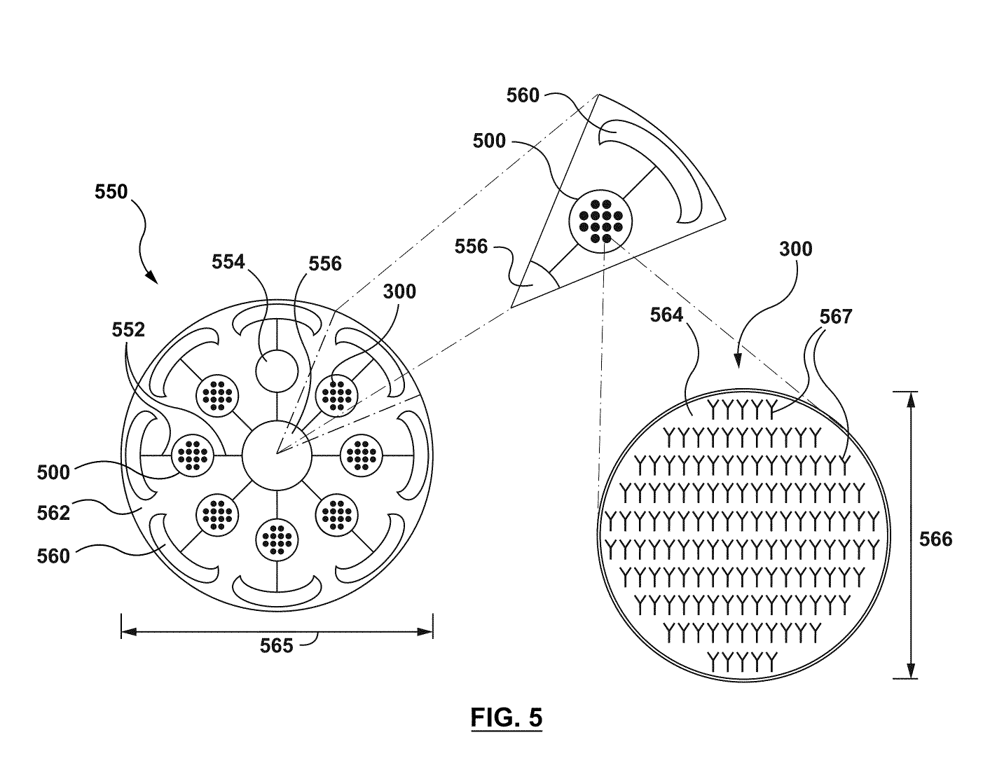

Referring now to FIG. 5, an embodiment of a microfluidic disk 550 is shown for combining a desired substance of a sample 120 with at least one amplification substance of a plurality of assemblies 300. Microfluidic disk 550 comprises a plurality of reaction chambers 500 each comprising a plurality of assemblies 300 disposed therein. Microfluidic disk 550 comprises microchannels 552, each of which extends from an approximately centrally located sample port 556 to provide fluid communication between a sample port 556, radially displaced reaction chambers 500, and overflow reservoirs 560 disposed at or near the periphery of the microfluidic disk. The sample port 556 may be shaped to receive a sample 120 retained by a collector 110, such as collector 410. The microchannels 552 provide fluid communication between overflow reservoirs 560, the reaction chambers 500 and the sample port 556. The microchannels may have mechanical or non-mechanical active and/or passive microvalves. The choice of amplification substances for each assembly depends on the target pathogen and detection modality. The term “disk” is used for clarity of illustration and is not meant to be limiting of the shape of the microfluidic disk 550; various shapes are contemplated. In an illustrative embodiment, the microfluidic disk 550 may have a diameter 565 of about 2 cm; each assembly 300 may have a diameter 566 of about 100 micrometers.

The microfluidic disk may have multi-layer construction comprising a top layer 562 extending above a bottom layer 564. The top layer and bottom layer may be mated along a mating surface. The top layer may be joined to the bottom layer along at least part of the mating surface, such as by bonding, or application of adhesive at least along the periphery. The microchannels, overflow reservoirs, and side walls of the reaction chambers may be fabricated into the top layer (e.g. by etching). In an embodiment, the top layer may be Polydimethylsiloxane (“PDMS”). Depending on the sample solution, the microchannels may be dimensioned and shaped to enable manual saliva supernatant separation and/or blood plasma separation from the desired substance of the sample via hydrodynamic filtration and Zweifach Fung effect. The bottom layer extends beneath the top layer. At least a portion of the bottom layer extending beneath the reaction chambers (and providing their surface) may be constructed similarly to conductive and insulating layers 310, 320 of the assembly of FIG. 3 (referred to as a “substrate layer”). Particularly, that portion may comprise an optically active substance (such as glass, silica, Sio2/Si, quartz, polymer, plastic, etc); and may comprise a layer of conductive substance (e.g. gold, silver or nanoparticles of gold or silver). Amplification substances relating to element 330 of assembly 300 for interacting with a desired substance of a pathogen may be immobilized/functionalized upon the substrate layer of the bottom layer as described above, providing assemblies 300 within the reaction chambers (such as pathogen monoclonal antibodies 567). In embodiments, the entire area of the bottom layer comprises an optically active substance, while the amplification substances may be disposed to be provided within the reaction chambers when the two layers are mated.

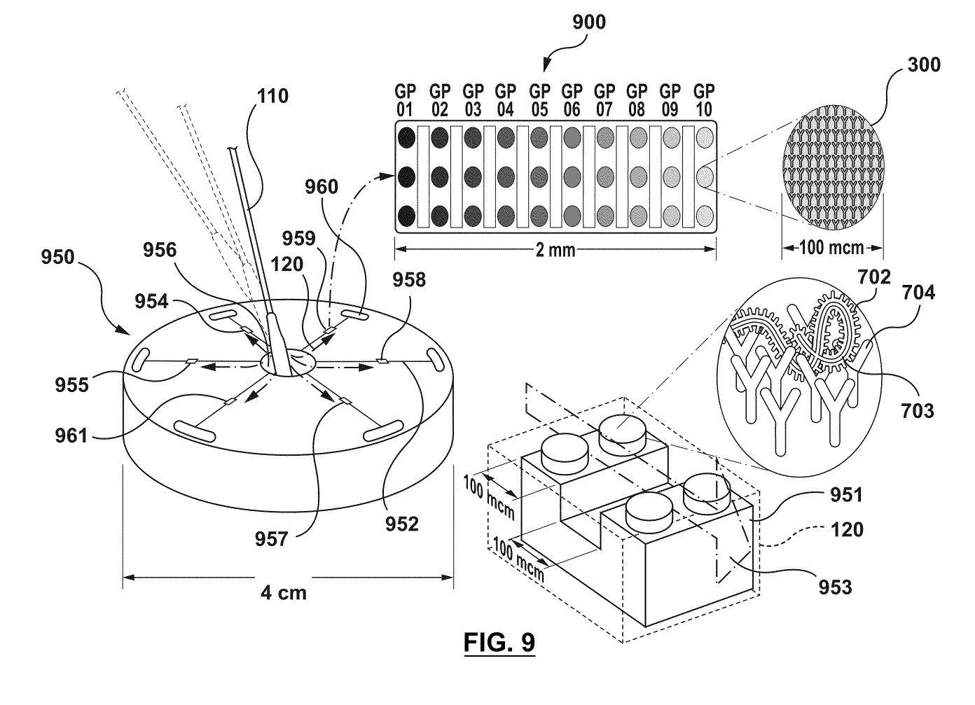

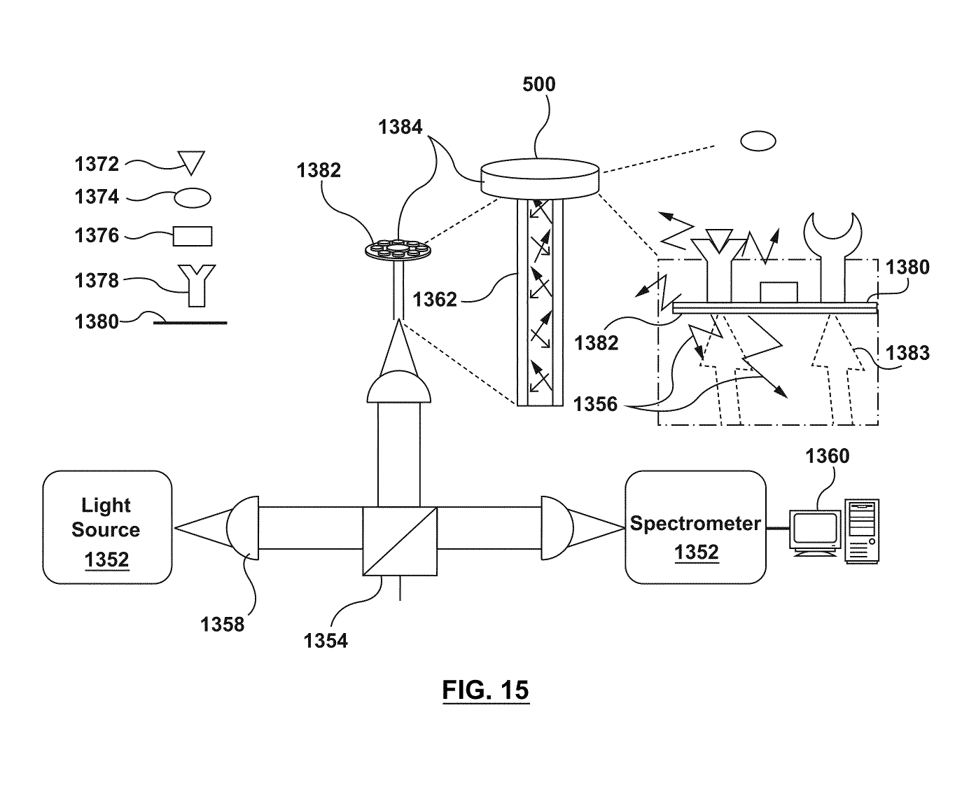

In embodiments, the bottom layer may further include a layer of protein NG or PEG, glycine, as shown and described in relation to FIG. 15 (at least at the location of the amplification substances), which may help to alleviate non-specific binding. The protein NG (or PEG, glycine) may be provided above the substrate layer; the amplification substances may be provided thereupon. Specifically, a protein NG layer may be applied first and then amplification substances (such as antibodies may be added). In the case of antibodies, the FC region of the antibody may attach to the substrate layer. Further, as described below in relation to FIG. 9 below, an optical Bragg grating may be provided. The Bragg grating may be made of Si on an SiO2 base, or of other dielectric materials, or a polymer. The protein NG or PEG, glycine layer may be provided atop the Bragg grating, such that the Bragg grating provides a base for the protein NG or PEG, glycine layer, and for the amplification substances provided thereupon.

In alternate embodiments, if the bottom layer is made of a substance that can support both being etched and receiving the amplifications substances, the microchannels, reaction chambers, and overflow reservoirs may be provided on a bottom layer, and amplification substances may be provided thereupon. In such embodiments, the top layer may merely comprise a lid comprising a sample port. The use of the terms “top” and “bottom” is illustrative, in some instances, the respective features may be flipped.

Amplification substances of assemblies 300 are selected for interacting with a desired substance of a target pathogen; accordingly, as shown in FIG. 6, for a microfluidic disk 550 directed to detection of environmental pathogens, E. Coli monoclonal antibodies might be provided as the amplification substance of at least one of the assemblies of at least one reaction chamber. As illustrated in FIG. 6, each reaction chamber 500 may comprise a different amplification substance to aid in the detection of target pathogen(s). FIG. 6 illustrates a microfluidic disk directed to detecting hemorrhagic fever 610, a bioterror disk 612, a disk for global infectious diseases 614, a disk for environmental pathogens 616, a disk for STIs 618, and a disk for oncology biomarkers 620. The hemorrhagic fever disk 610 may include Ebola mAbs, Lassa mAbs, and Marburg mAbs. The bioterror disk 612 may include Anthrax mAbs, small pox mAbs and plague mAbs. The global infectious disease disk 614 may include TB mAbs, malaria mAbs and Dengue mAbs. The environmental pathogens disk 616 may include E. Coli mAbs, Cryptosporidium mAbs and Salmonella mAbs. The STI disk 618 may include HIV mAbs, HSV mAbs and Treponema pallidum mAbs. The Oncology biomarkers disk 620 may include CA-125 mAbs, BRCA1/BRCA2 mAbs, and CA 19-9 mAbs. Each microfluidic disk comprises a control reaction chamber 554. Each individual reaction chamber 500 may be equipped with one of various assemblies, each having different amplification substances. For example, in FIG. 5, each reaction chamber is shown to comprise twelve assemblies having different functionalized bioreceptors (such as monoclonal antibodies). Further, each assembly may comprise microarrayed amplification substances. Accordingly, the reaction chambers may provide multiplexed biosensors, which aid in ensuring binding in view of pathogen mutation, pleomorphism and polymorphism. For example, each reaction chamber 500 may comprise different antibodies to detect different glycoproteins of a pathogen.

In the illustrated embodiment, the reaction chambers 500 and assemblies 300 are shown to be shaped approximately circularly. Other configurations are contemplated to optimize the spread of the sample within the reaction chambers. Particularly, the reaction chambers may be shaped with a narrower end radially proximal to the port 556. Additionally the reaction chambers, microchannels and reservoirs may be disposed on the microfluidic disk 550 optimally given the flow characteristics of the sample under the centrifugation from the port 556. Particularly, the microchannels may be disposed in a spiral pattern extending outwardly from the sample port.

In embodiments, one of the reaction chambers 500 may be reserved as a control reaction chamber 554. The control chamber 554 may not be fluidly linked to the sample port 556, to prevent flow of the sample 120 thereto. The control chamber 554 may serve to provide control reference signals during detection operations, wherein any detection signal received from detection operations performed on the control chamber indicates an absence of the binding of a desired substance.

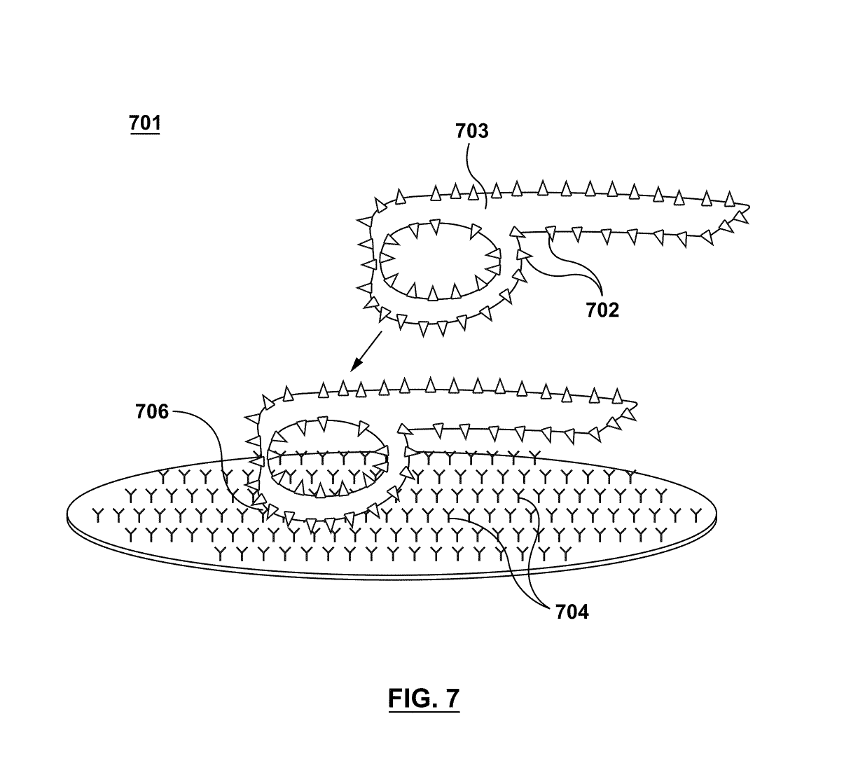

In use, a sample 120 may be deposited by a collector 110 at the sample port 556 and the microfluidic disk 550 may be centrifuged to cause the sample 120 to flow through the microchannels 552 to reaction chambers 500, causing desired substances in the sample 120 to bind to amplification substance(s) of each assembly 300. Once the sample is deposited, the microfluidic disk 550 may be centrifuged for a predetermined, user-configurable, or automatically determined amount of time (and rotational velocity). Excess flow passing through a reaction chamber may flow to overflow reservoir 560. As illustrated in FIG. 7, once the sample 120 arrives at each of the chambers 500 (providing a saliva matrix 701), a desired substance (such as a glycoprotein 702 of Ebola virus 703) from the sample will bind to the amplification substance (such as a glycoprotein antibody 704) in the reaction chamber, the binding of which (see element 706 viral-antibody binding) may be subsequently detected by detections operations 220 described herein.

In an example wherein the disk 550 is used with the collector 410 described in relation to FIG. 4, the fluid collector 422 end of the collector 410 is placed within the sample port 556, which may cause the sample to at least partly deposit onto the microfluidic disk. The sample may further release upon partly depressing the plunger 412. Once the sample is released, the microfluidic disk 550 may be centrifuged to spread the sample through the microchannels. The collector 410 may be removed from the sample port 556 during centrifugation. Preferably after centrifugation, the fluid collector 422 may then be repositioned into the sample port 556 and the plunger 412 may be fully depressed, causing the rupturable buffer chamber 418 to rupture, releasing a buffer solution, and washing away any non-binding substances. It will thus be appreciated that the buffer solution may serve to clean away saliva, and any other substance that hasn't bound to the amplification substance of the various assemblies, as described above.

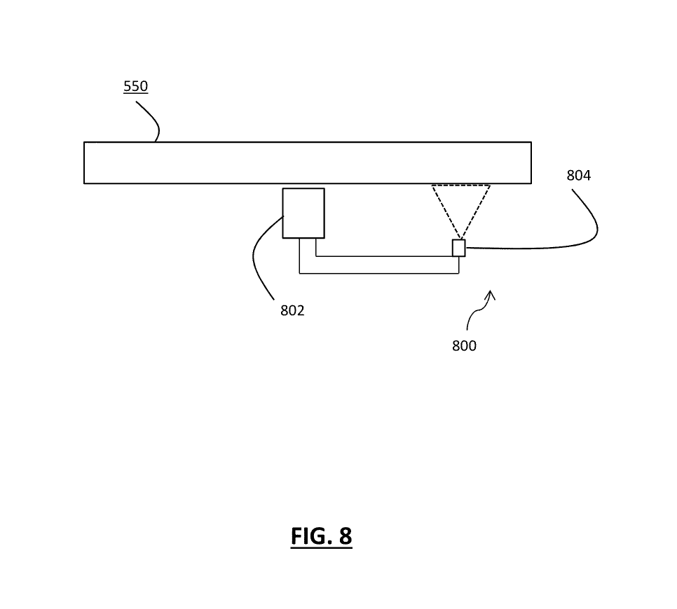

Referring now to FIG. 8, a centrifugation unit 800 may be provided to control the length and rotational properties of centrifugation. The centrifugation unit comprises an actuator 802 mechanically coupled to the microfluidic disk 550 for centrifuging the microfluidic disk, a controller, and may comprise a monitoring module for monitoring spread of the sample 120 to each of the reaction chambers 500. The actuator may be a rotationally controllable motor, such as a stepper motor. The monitoring module comprises or is communicatively linked to at least one detector 804, such as an optical sensor unit, for generating signals for processing by a processor (optionally of the computing device) to monitor spread of sample 120 over at least one chamber (or assembly) of the microfluidic disk during centrifugation. Once the processor determines that the flow has spread to encompass a predetermined percentage of the surface of at least one chamber 500, such as at least 75% of the surface of a particular chamber, rotation may be stopped. The centrifugation unit can include a controller having appropriate learning mechanisms to calibrate the control of centrifugation based on historical detector data. The learning mechanisms may moderate the speed and duration of centrifugation based on feedback from the processor relating to the spread of the sample and the relative success of subsequent detection operations. For example, if detection operations are unsuccessful after a particular centrifugation configuration (i.e. detection signals don't conform to expected results), or if sample spread is insufficient, rotational velocity of centrifugation may be adjusted or even intermittently reversed. Though described in relation to the microfluidic disk 550, the centrifugation unit may be used in conjunction with other embodiments described herein where centrifugation of the sample is required during processing.

Referring now to FIG. 9, shown therein is another embodiment of the microfluidic disk. The illustrated microfluidic disk 990 similarly comprises reaction chambers 900, sample port 956, microchannels 952 and overflow reservoirs 960 provided in a top layer. In the illustrated embodiment, each reaction chamber 900 is shaped rectangularly in cross-section and is provided with an array of assemblies comprising amplification substances (provided on the bottom layer). In an illustrative embodiment, the reaction chambers may include Zaire Ebola GP Antibodies 954, Sudan Ebola GP Antibodies 955, Lassa GP antibodies 956, a control chamber 957, Marburg GP Antibodies 958 and another type of amplification substance at 959. The embodiment of FIG. 9 relates to an example embodiment of the microfluidic disc comprising a Bragg grating 951 that may be utilized with certain detection modalities where it may be desired to cause incident light to generate an evanescent wave (illustrated as 953), which may be required according to some detection modalities, such as some interferometric detection modalities.

As described in the RP PhotonicsEncyclopedia at https://www.rp-photonics.com/bragg_gratings.html, an optical Bragg grating is a transparent device with a periodic variation of refractive index, so that a large reflectivity may be reached in some wavelength range around a wavelength which fulfills the Bragg condition

$$\frac{2 π}{Λ} = 2 \cdot \frac{2 π n}{λ} cos θ$$

where λ is the vacuum wavelength of light, n the refractive index, θ the propagation angle in the medium relative to the direction normal to the grating, and Λ the grating period. If fulfilled, the wavenumber of the grating matches the difference of the wavenumbers of the incident and reflected waves.

In the embodiment of FIG. 9, on a bottom layer, the assemblies 300 are provided with amplification substances (such as functionalized antibodies) on a grating 951 positioned atop a substrate layer (and further optionally atop a protein NG layer). The embodiment will undergo a change in refractive index upon pathogen binding to the amplification substances of the assemblies. One may or may not use a collimated white light input that may require the biosensor to create a change in the Bragg diffraction condition such that the wavelength shift is at least 5 nm. As seen in the microfluidic disk 950, each area of functionalized antibodies that act as bioreceptors for pathogen, biomarker targets may, in some example embodiments, measure approximately 100 micron spots diameter on a 200 micron pitch over a 4 mm height.

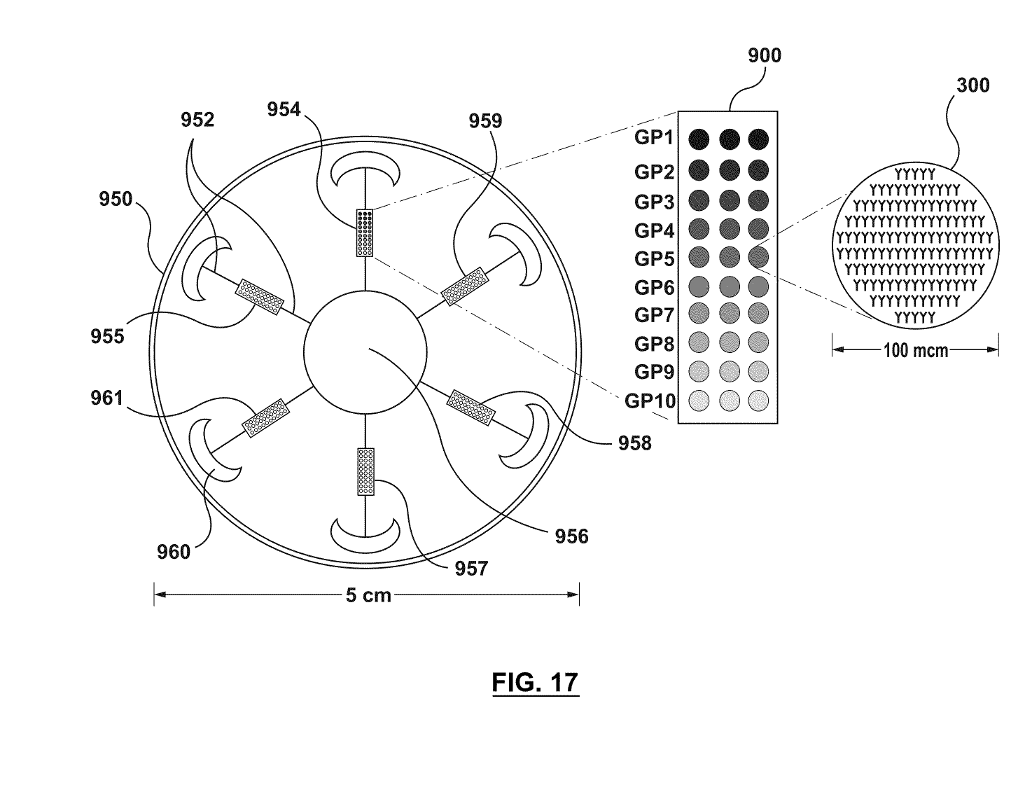

FIG. 17, provides another view of the microfluidic disk of FIG. 9.

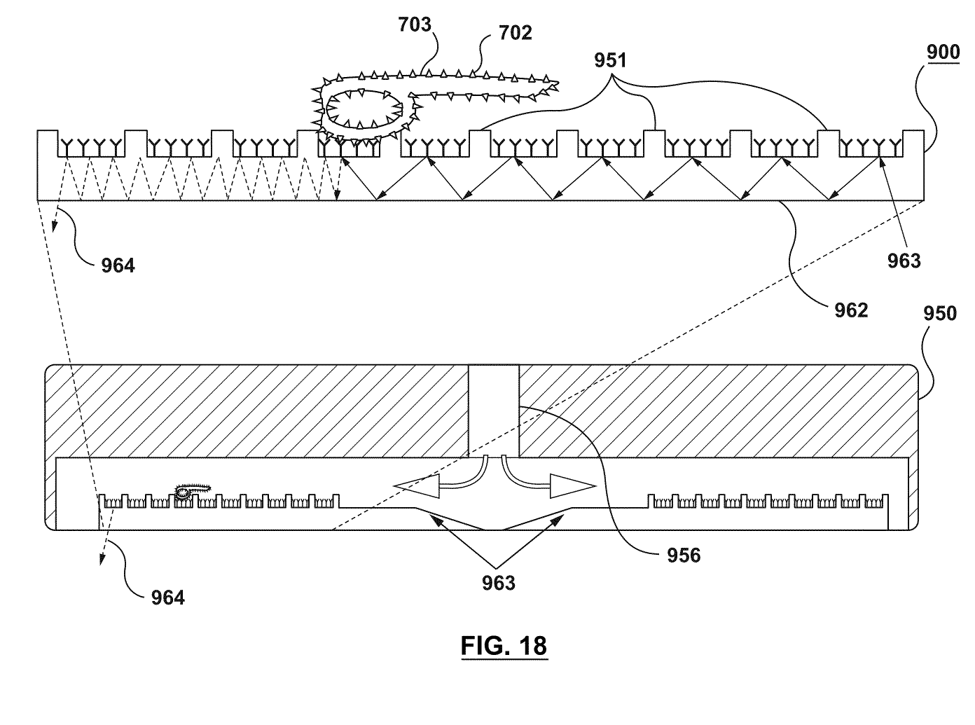

Referring now to FIG. 18, a waveguide may be provided in the microfluidic disk to direct light during detection operations, as required for some detection modalities and some configurations of the microfluidic disk or detector. Particularly, as shown in FIG. 18, in some embodiments light may be received centrally along the bottom of the microfluidic disk; a waveguide may be provided as illustrated to direct light to the peripheries of the microfluidic disk where it may be received for generating detection signals (such as by a spectrometer). Embodiments of the microfluidic disk (e.g. 550, 950) may include a single mode or multimode optical planar waveguide and include thin film deposition techniques/application as described below.

As described in https://en.wikipedia.org/wiki/waveguide_(optics), a waveguide may be provided in order to guide electromagnetic waves. At optical frequencies waveguides may include a dielectric material with high permittivity surrounded by a material with lower permitivity. Waves may accordingly be guided by total internal reflection. Practical rectangular-geometry optical waveguides may be considered as variants of a theoretical dielectric slab waveguide, also called a planar waveguide. The slab waveguide consists of three layers of materials with different dielectric constants, extending infinitely in the directions parallel to their interfaces. Light may be confined in the middle layer by total internal reflection if the dielectric index of the middle layer is larger than that of the surrounding layers. In practice, if the typical size of the interfaces is much larger than the depth of the layer, the slab waveguide model is a good approximation. Guided modes of a slab waveguide can not be excited by light incident from the top or bottom interfaces. Light can be injected with a lens from the side into the middle layer. Alternatively a coupling element may be used to couple light into the waveguide, such as a grating coupler or prism coupler. One model of guided modes is that of a planewave reflected back and forth between the two interfaces of the middle layer, at an angle of incidence between the propagation direction of the light and the normal, or perpendicular direction, to the material interface is greater than the critical angle. The critical angle depends on the index of refraction of the materials, which may vary depending on the wavelength of the light. Such propagation will result in a guided mode only at a discrete set of angles where the reflected planewave does not destructively interfere with itself.

As described in Waveguide-Based Biosensors for Pathogen Detection, Harshini Mukundan, http://www.ncbi.nlm.nih.gov/pmc/articles/PMC3274158/, a single mode planar waveguide can support several thousand reflections per centimeter of beam propagation for visible wavelengths, two orders of magnitude higher than multimode planar and fiber waveguides. Single planar waveguides can also enable the rapid decay of the evanescent field away from the waveguide surface with no appreciable intensity beyond one-half the wavelength of the excitation light (˜250-300 nm). As a result, the strong spatial filtering effect inherent in single mode planar waveguides has the ability to enhance sensitivity by minimizing background from interferents and allow direct analysis of complex samples while eliminating the need for additional rinsing and drying steps with a buffer. However, this increased sensitivity for single mode waveguides requires certain modifications of the waveguide such as thin film deposition and use of grating couplers to couple excitation light into the waveguide films. Typically comprised of a very thin (<wavelength of excitation) high dielectric index film such silicon oxynitride or tantalum pentoxide and others are deposited on a low index substrate. Alternative fabrication approaches may or may not include the use of sol gels and ion deposition methods.

Referring now to block 220 of method 200, once a sample has been processed as desired, and once the processed sample has been combined with any amplification substance (such as by depositing a sample onto an assembly 300), detection operations can be performed by detector 130. The detection operations generate detection signals that can be processed at device 140 or remote computers 160 to determine the presence of a desired substance indicating the presence of a pathogen in the sample. Detection signals vary depending on whether a desired substance is present in the sample 120, and more particularly whether a desired substance has bound to an amplification substance. In embodiments comprising a microfluidic disk 550, at least one detection signal may be provided for each of the reaction chambers 500. Further, where each reaction chamber 500 comprises a plurality of amplification substances, various detection signals may be received for different sections of each reaction chamber 500. In various embodiments described herein, the detection operations can be performed to receive detection signals from the bottom surface of the assembly (or reaction chamber). However, in some embodiments, as long as detection signals can be received that can be analyzed to determine the presence or absence of a desired substance, the detection operations can be performed from other directions, such as from the top of the assembly (or reaction chamber).

Detector 130 can take any form that is suitable for the detection of the desired substance. Accordingly, various detection modalities are contemplated. For example, the detector 130 can be a molecular sensing array designed to be sensitive to one or more desired substances. Further, the detector 130 can be configured to perform Surface-Enhanced Raman Spectroscopy (SERS), Surface Plasmon Resonance (SPR), Surface Plasmon Resonance Imaging (SPRi), Localised Surface Plasmon Resonance (LSPR), Optofluidic Nanoplasmonic, Optical waveguide-based sensing, Optical ring resonator-based sensing, Photonic crystal-based sensing, Nanosensitive OCT sensing, Lensless digital holographic imaging, Superresolution microscopy techniques, piezoelectric sensing, nano-cantilever sensing, Raman spectroscopy (RS), Resonance Raman spectroscopy (RRS), and infrared spectroscopy (IRS). In variations, the detector 130 can be configured to perform interferometer-based detection, such as by using a Mach-Zehnder Interferometer, Young's interferometer, Hartman interferometer, interferometric scattering microscopy (iSCAT), Single Particle interferometric Reflectance Imaging (SPIRIS) and backscattering interferometry. The detector 130 can have physical dimensions that range from approximately 5 centimeters (cms) on each side to two meters on each side. Other detectors based on other detection techniques will now occur to a person of skill and are contemplated.

A spectrometer typically operates by illuminating a sample with one or more specific wavelength radiation (e.g. one or more source radiation), and by detecting the resulting emission or scattering of energy produced as detected spectral signals. By analyzing the range of amplitudes and wavelengths in the detected spectral signals the substances contained in the sample 120 can be identified. For example, Raman spectroscopy is a spectroscopic technique for detecting vibrational, rotational and other low-frequency modes and is based on a scattering of monochromatic source radiation such as that produced by a laser. The source radiation can be in the visible, near infrared or near ultraviolet range. The source radiation produced by the detector 130 can interact with molecular vibrations or other excitations in the sample 120, resulting in the energy of the photons being changed, and in turn yielding a Raman spectral signal. The Raman spectral signal can thus provide information about the substances present in the sample 120. For example, in some variations, a region of the sample 120 can be illuminated with a laser. The resulting radiation from the illuminated region can be collected with a lens and sent through one or more filters and dispersed onto a sensor, resulting in a Raman spectral signal.

As another example, Resonance Raman (RR) spectroscopy is a type of vibrational Raman spectroscopy in which the source radiation frequency is close to an electronic transition of the desired substance to be detected in the sample 120. The resulting frequency resonance can enhance the intensity of the Raman spectral signal which can facilitate the study of small samples 120. For example, in the present example, an excitation wavelength of 785 nm and/or 681 nm and 5% maximum (450 mW) laser power can be used.

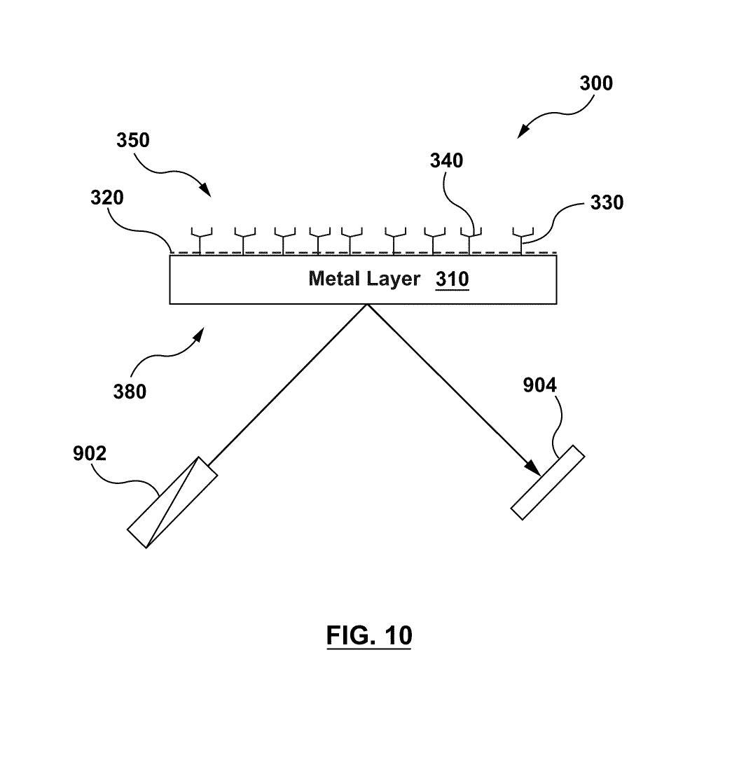

As a further example, a surface plasmon is an electro-magnetic wave propagating along the surface of a thin electrically conducting layer, such as a metal layer. Surface plasmon resonance (SPR) is achieved through the generation of electron charge density waves based on an incident radiation. The intensity of reflected light can be reduced at a specific angle, known as the resonance angle. Accordingly, to perform SPR, a source radiation 902, such as an electron or light beam is provided to a metal surface at an angle, such as the bottom surface 380 of the assembly 300 as shown in FIG. 10. The reflected radiation is subsequently received at a sensor 904 which yields an SPR signal. At specific angles of the radiation source, the excitation of surface plasmons can take place, reducing the intensity of the reflected light. The specific angles at which the reduction takes place can vary based on various properties of the assembly 300. Accordingly, a slight change at the metal surface can lead to a change in SPR signal, allowing precise measurements of thin film properties as well as surface molecular interactions. Thus, the SPR signal can vary based on the density of the attached antibodies 330 as well as whether any of the attached antibodies 330 are bound to the desired substance.

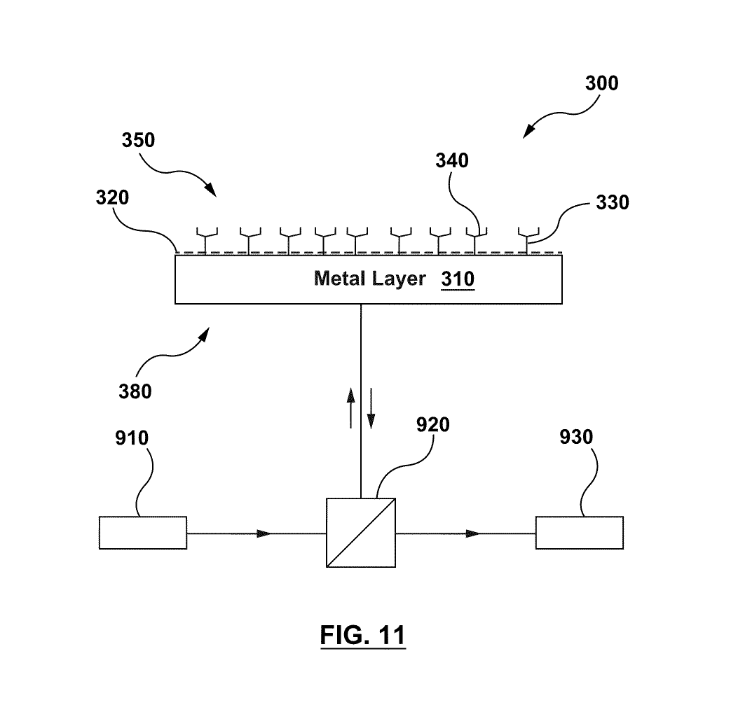

In yet a further example, FIG. 11 illustrates application of backscattering interferometry for detection. The illustrated embodiment ignores the use of lenses and other components, the needs for which would be understood by a person of skill. Backscattering interferometry may be effected by providing coherent source radiation emitted from a source 910 through a beam splitter 920, which directs the source radiation to both a surface of a sample, such as via the bottom surface 380 of the assembly 300, and a sensor 930, as illustrated. The source radiation is backscattered from the sample and is refracted by the beamsplitter 920 toward the sensor 930, which consequently obtains an interference signal comprising both the source radiation and the backscattered radiation. The interference signal contains information relating to the unique backscattering of the incident radiation resulting from binding of desired substances to the assembly 300 and resulting sub-wavelength structures, providing reflected radiation having a modulated phase, and resulting in interference at the sensor. Detection of the backscattering at the sensor thus relates to the backscattering signal having a unique intensity. Typically in backscattering interferometry, the source radiation is provided to a small surface of a sample, and the sample may be housed in a microfluidic channel to introduce reflection of incident radiation in the channel cavity. The sensor may comprise a charge-coupled-device camera (CCD) camera. The source radiation may be supplied by a fixed wavelength laser, such as a helium-neon laser.

In some variations, the detector 130 can allow different detections techniques and/or variations in a detection technique to be combined, thus allowing the system 100 to take advantage of the fact that different detection techniques and/or variations in the same technique can yield similar but complementary information, potentially increasing the detectability of the desired substance. The different techniques and/or variations in the detection techniques can be performed simultaneously, sequentially or both. For example, in some variations, the detector 130 can include the appropriate components (radiation sources and sensors, for example) to perform two or more different detection techniques such as performing IRS as well as RRS and SPR. In other variations, the detector 130 can include the appropriate components to perform variations of the same detection technique. For example, the detector 130 can include multiple radiation sources such as multiple fixed wavelength lasers able to generate source radiation at different wavelengths. Alternatively, the detector 130 can include a single tunable radiation source such as a tunable laser that is also able to generate source radiation at different wavelengths. Accordingly, the same spectroscopy technique can be performed at various wavelengths on the same sample. As a further example of varying a detection technique, the same detection technique can be performed at different temperatures. For example, the detector 130 can include a heating element such as an infrared laser operable to change the temperature of the sample 120.

In some variations, the different detection techniques and/or variations of the same detection technique can be applied to processed and/or unprocessed samples 120. For example, in some variations, some of the various detection techniques or variations of the same detection technique can be applied to the sample 120 that is combined with the amplification substances. Alternatively, some of the various detection techniques or variations of the detection techniques can be applied to the sample 120 that is not combined with the amplification substances. In yet further variations, the different detection techniques and/or variations of the detection techniques can be applied to materials or solutions containing the amplification substances alone, such as prefabricated test materials, which may provide detection signals indicating the absence of the desired substance.

To perform different detection techniques, in some variations, the detector 130 can comprise of various separate detectors. For example, where IRS as well as RRS are performed sequentially on the sample 120, different spectrometers can be used to perform the IRS and the RRS techniques. In these cases, the sample 120 can be divided into multiple samples, each of the divided samples being provided to one of the separate detectors.

The following process is a non-limiting illustrative example of combining detection techniques and technique variations. Initially, RS or SPR can be performed on the sample 120 at room temperature prior to combining it with amplification materials. Following the initial detection, the sample 120 can be combined with the amplification materials forming a combined sample 120. After a pre-determined incubation period, RS or SPR can be once again performed at room temperature, on the combined sample 120. The performance of the RS or SPR can be repeated on the combined sample 120 at 110 degrees Centigrade. The temperature of the combined sample 120 can be set, for example, through the use of an infrared laser or a cooler. Upon completion of the RS scan and SPR at 110 centigrade, RRS can be performed on the combined sample, once the sample is cooled back to the room temperature. The RRS may be performed using variations, specifically at the wavelengths of 400 nanometers (nm), 450 nm, 600 nm and 1000 nm, using four different source lasers.

The combination of detection techniques and technique variations can be pre-determined or static. Thus the same combination is applied to each sample 120. The above discussed non-limiting example of the detection technique combination is an example of a pre-determined static combination. In variations the combination can be dynamic. For instance, the dynamic nature of the combination can be based on the detection signal acquired based on the previous detection operation performed. Accordingly, the detection signal obtained based on a particular detection operation, such as RS or SPR, can determine the next detection operation to be performed. For example, in some variations, certain detection operations can only be applied when the presence of certain substances are detected based on previous detection operations. As a further example, for each sample 120, the detection can start by combining the sample 120 with the amplification materials forming a combined sample 120. After a pre-determined incubation period of 5 seconds, RS or SPR can be performed at room temperature, on the combined sample 120. The performance of the RS or SPR can be repeated, every five seconds, until a Raman spectral signal or SPR signal is obtained that includes sufficient information for a detection analysis to be performed. Other variations for determining the dynamic nature of detection technique combination will now occur to those of skill and are contemplated.

Referring now to block 230 of method 200, once one or more detection operations are performed at block 220, the received detection signals are analyzed at the device 140 (and/or 160) at block 230 of method 200. The analysis can be based on various methods. For example, two different reference signals can be identified, the first one indicating the absence and the second one the presence of the desired substance. Accordingly, signals obtained for each sample 120 can be compared to the reference signals, and a determination can be made as to whether the desired substance is present in the sample. The determination can then be indicated to a user of the device.

As a non-limiting illustration, in the present example, the sample 120 includes the glycoproteins ZEBOV-GP as the desired substance and the ZEBOV-GP antibodies as the amplification substance, the antibodies including gold nanoparticles bound to them as the enhancer substance. Applying RS to the combined sample 120 of the present example can result in two potentially different Raman spectral signals which can be identified as the two reference signals. A first reference signal results from antibodies that are not bound to the glycoproteins. A second reference signal results from the antibodies that are bound to the glycoproteins. Accordingly, when Raman spectral signals obtained from a combined sample 120 are analyzed, a determination can be made as to whether the combined sample 120 includes glycoproteins or whether the glycoproteins are absent. Specifically, the obtained Ramen spectral signals can be compared with the two reference signals to make a determination. The determination that the sample 120 includes the glycoproteins, in turn, indicates the presence of an Ebola infection with the sample provider.

As a further non-limiting illustration, in accordance with the example of FIG. 3 and FIG. 10, the sample 120 includes the glycoproteins ZEBOV-GP as the desired substance and the ZEBOV-GP antibodies as the attached amplification substance 330. SPR can be applied to the assembly 300 as shown in FIG. 10 by applying a source radiation 902 to the bottom side 380 of the assembly 300. Applying SPR to the assembly 300 as shown in FIG. 3 can result in two potentially different SPR signals which can be identified as the two reference signals. A first reference signal results from an assembly 300 where the attached antibodies 330 are not bound (or the percentage bound is below a threshold) to the glycoproteins. A second reference signal results from an assembly 300 where the attached antibodies 330 are bound (or the percentage bound is above a threshold) to the glycoproteins. Accordingly, when SPR signals obtained from a combined sample 120 are analyzed, a determination can be made as to whether the combined sample 120 includes glycoproteins or whether the glycoproteins are absent. Specifically, the obtained SPR signals can be compared with the two reference signals to make a determination. The determination that the sample 120 includes the glycoproteins, in turn, indicates the presence of an Ebola infection with the sample provider.

As yet a further non-limiting illustration, in accordance with the example as illustrated in FIG. 11, backscattering interferometry can be performed by applying a source radiation 910 to the bottom side 380 of the assembly 300. As above, an interference signal can be detected by sensor 930 depending on whether the desired substance has bound to the assembly 300. Accordingly, when backscattering interferometry is performed on a combined sample, signals from sensor 930 are analyzed to provide a determination as to whether the combined sample 120 includes the desired substance (e.g. glycoproteins) or whether the desired substance is absent.

The identification of the reference signals, which can be more than two or less than two, depending on the detection technique or techniques used, can be based on detection signals and various methods. In some variations, the identification can be made manually, by performing the detection operations on the sample 120 in the presence and absence of the desired substance, and selecting the appropriate signals as reference signals. In variations, the identification can be made automatically based on various automated learning algorithms such as supervised, semi-supervised and unsupervised learning algorithms, through the use of neural networks or clustering mechanism, for example. Neural networks used can be probabilistic. In some variations, the same mechanisms used for automatically identifying the reference signals can also be used to perform the signal match analysis. For example neural networks or clustering mechanism used for identifying reference signals can also be used for performing the matching of a detected signal to one of the reference signals. In yet further variations, there may not be separate reference signal identification process. Instead, learning based mechanisms, such as neural networks and clustering mechanism can learn to detect the presence or absence of a desired substance based on the detected signals, employing various learning schemes. In some further variations, the learning mechanisms can be primed with unsupervised data so that they are primed for the detection of the presence or absence of the desired substance based on detection signals received from the detector 130.

In variations, the identification of reference signals, or the detection of the presence and absence of a desired substance using neural networks or clustering mechanisms can be an ongoing process. For example, in some implementations, the device 140 can provide the results to the remote computers 160. The remote computers 160 can include appropriate learning mechanisms to update the reference signals based on the newly received signals. For example, the remote computer 160 can be a neural network based system implemented using various application programming interfaces (APIs), and can be a distributed system. The APIs included can be workflow APIs, match engine APIs and signal parser APIs, allowing the remote computer 160 to both update the network and to determine whether a match is detected based on the received detection signal.

The use of neural networks as described above may facilitate the use of interferometry-based detection modalities by overcoming quantum realm challenges traditionally associated therewith, as described in embodiments provided below. Particularly, the use of neural networks for processing signals from sensor 930 allows for additional inference over traditional detection modalities.

In further variations, the analysis of the detection signals as well as identification of the reference signals can include additional data obtained from sources other the detector 130. For example, thermal imaging signals, sample provider history such as locations visited and flight information and other data can be included in the analysis (and, where appropriate as part of the reference signal), in addition to the detection signals from the detector 130. For example, in the present example, when the detection signal for a sample 120 is matched to a reference signal indicating the presence of the glycoprotein with a weak confidence level, a thermal image indicating a fever may be used to increase the confidence level of the match. As a further example, the detection signal can include travel pattern of the sample provider, thus allowing the system 100 to take into account the sample provider's travel history in determining a match, and thus the presence or absence of the desired substance.

In some implementations, the method for detecting a desired substance can be varied such that multiple desired substances can be detected, optionally for more than one of the pathogens described above. For example, multiple amplification substances can be included, each targeted at a different one of the desired substances. The analysis performed can then identify multiple reference signals for determining the presence or absence of one or more of the desired substances. More complex analysis, such as those based on clustering methods and neural networks can also be used to differentiate between the different substances based on one or more detection signals obtained on the basis of a sample 120.

Referring now to block 240 of method 200, once the detection operations 220 are complete, and optionally prior to analysis 230 of the detection results, the assembly 300 may be provided for decontamination. The disposal unit may comprise an incinerator or an autoclave.

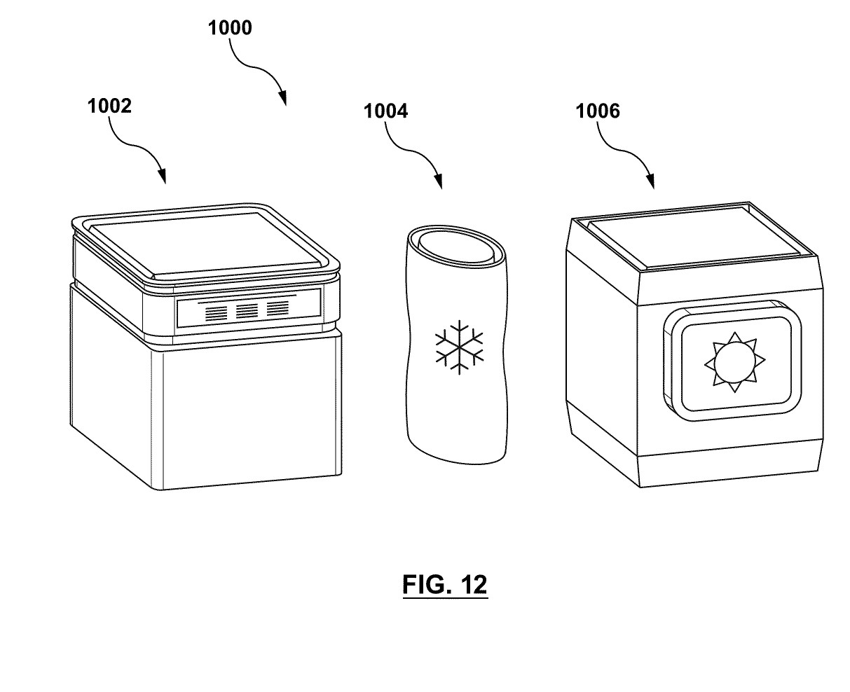

Referring now to FIGS. 12 to 16, apparatuses for pathogen detection are shown. Particularly, the apparatuses of FIGS. 12 to 16 comprise components for effecting the steps of method 200.

Referring first to FIG. 12, the apparatus 1000 comprises a detector unit 1002, a storage unit 1004, and a disposal unit 1006.

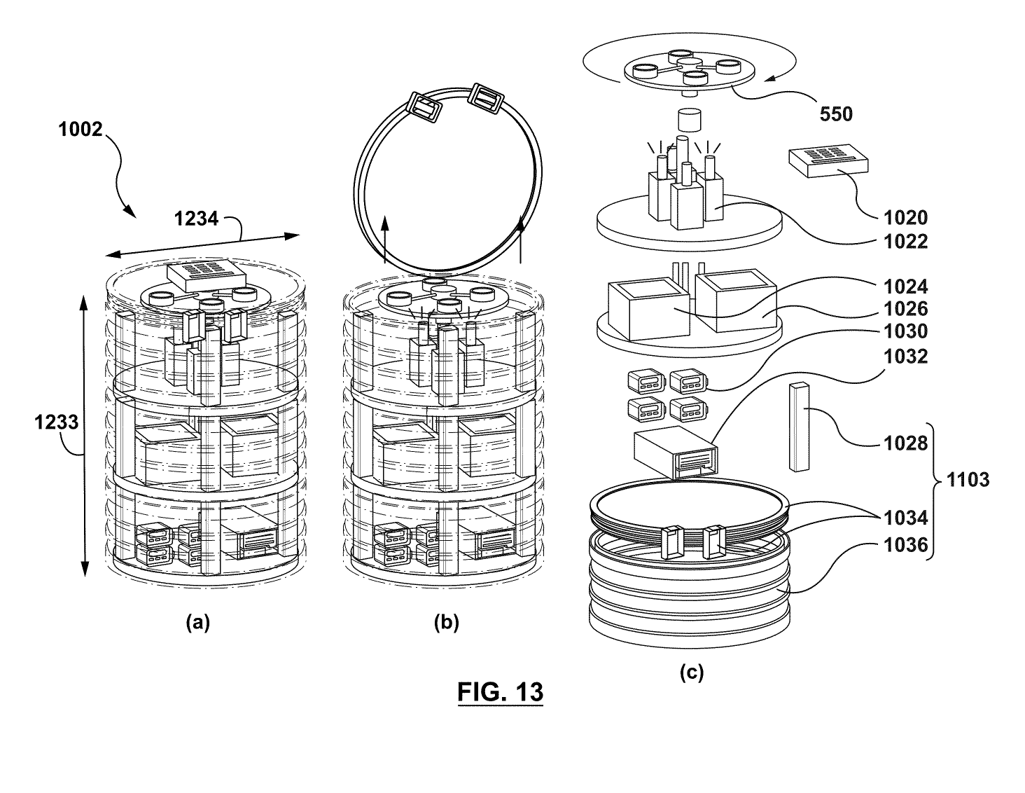

The detector unit 1002 is adapted to receive a sample 120 from a collector 110, preferably provided on a microfluidic disk (such as disk 550), and perform detection operations 220 and analysis 230 thereupon. The detector unit 1002 accordingly comprises components for receiving the sample, and performing detection and analysis thereon. Particularly, referring to FIG. 13, according to a particular embodiment the detector unit 1002 comprises at least controls 1020, optical fibers and beam splitters 1022, a spectrometer 1024, a photon source 1026, a power supply 1030, and a CPU 1032. Further, to provide a rigid casing 1103, the detector unit comprises a plurality of reinforcement bars 1028, as well as a durable reinforced shell 1036 and durable hatch and latches 1034. In some embodiments, the detector unit may be about 20 cm tall (shown as element 1233), and 10 cm in diameter (shown as element 1234).

The storage unit 1004 provides storage for a plurality of microfluidic disks 550, each comprising a plurality of reaction chambers 500 with amplification substances for detecting at least one pathogen, as described above. The storage unit may have refrigeration hardware for maintaining the microfluidic disks 550 at a particular temperature to avoid degradation of the effectiveness of the amplification substances. Optionally, the storage unit 1004 comprises a plurality of sections having differential temperature control, which may be used for microfluidic disks 550 having different preferred storage temperatures to avoid degradation of particular amplification substances. For clarity of illustration, and not by way of limitation, embodiments relate to the use of the microfluidic disk 550 for providing a sample to the apparatus. With necessary modifications, a sample could instead be provided to the apparatus 1000, for example, on a single assembly 300, or in a vial.

The disposal unit 1006 comprises disposal equipment, such as an incinerator or an autoclave, for disposing of a sample once detection and analysis are completed. Particularly, the disposal unit 1006 may be connected to a control system controlling the disposal equipment therein for sterilizing a sample upon receiving a signal indicating request from a user has been received. If the sample is provided on a microfluidic disk 550, the disposal unit 1006 is configured to receive a microfluidic disk 550 comprising a plurality of reaction chambers 500.

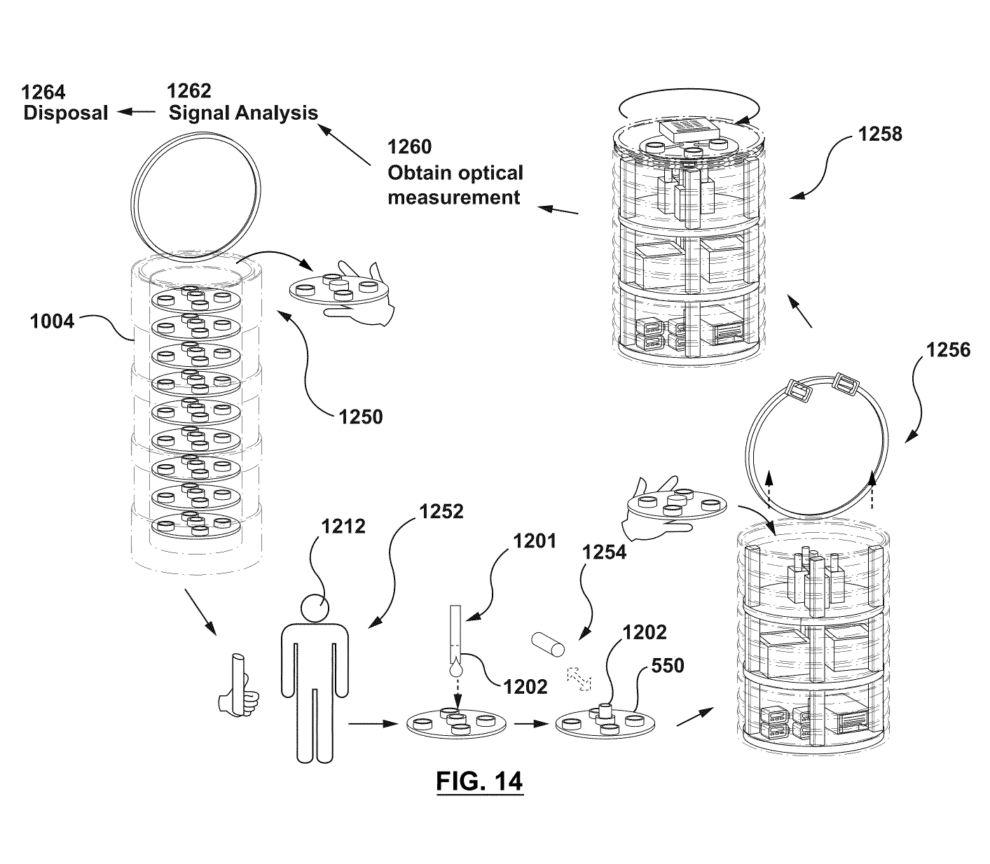

As illustrated in FIG. 14, to effect the steps of method 200 at the apparatus 1000, the detector unit 1002 receives a sample 120, which may be provided on a microfluidic disk (such as disk 550), and performs detection operations 220 and analysis 230 thereupon. Illustrated blocks 1250 to 1258 correspond to block 210 of method 200. At block 1250, the microfluidic disk 550 may be collected from the storage unit 1004. At block 1252, a sample is collected on a collector from the subject 1212. At block 1254, the sample is deposited to the microfluidic disk 550.

With respect to blocks 1252, 1254, in FIG. 14, an embodiment of a collector 1201 is illustrated for collecting a sample and providing the sample 120 to the microfluidic disk 550. In use, a sample may be provided at a fluid collecting end 1202 of the collector 1201; the fluid collecting end 1202 of the collector 1201 can be placed into a sample port 556 of the microfluidic disk 550; and the fluid collecting end 1202 can be broken off from the collector 1201, depositing the sample for centrifugation. The collector 1201 may be perforated to allow the collector 1202 to be easily broken off.

At block 1256, the microfluidic disk 550 will be placed into the detector unit 1002 so that processing and detection can be carried out. At block 1258, the microfluidic disk 550 can be processed by centrifugation, as described above, to distribute the sample to the reaction chambers 500. The microfluidic disk may be centrifuged for a predetermined length of time, such as twenty to thirty seconds. Optionally, a buffer solution may be applied to clear away non-specific substances remaining after centrifugation. At block 1260, corresponding to block 220, detection signals can then be received from each of the reaction chambers 500. An illustrative detection operation is shown in FIG. 15, wherein a light source 1352 provides light through an optical splitter 1354 which is then backscattered from a reaction chamber 500 to the splitter, providing a detection signal to a spectrometer 1354. At block 1262, the signals can then be processed, either locally by the CPU of the detector unit 1002, or by providing the signals over a communication network for remote analysis, as described above. At block 1264, the sample, and the microfluidic disk 550, can be disposed of in the disposal body 1006.

Referring again to the illustrative detection operation of FIG. 15, more particularly, a detection operation is shown wherein a sample 1384 is provided to a reaction chamber; a light source 1352 provides radiation through an optical splitter 1354 and an optical fiber 1362 to the reaction chamber (as incoming light 1383) which is then backscattered (shown as element 1356) to the splitter, providing a detection signal to a spectrometer 1352 for analysis by a computing device 1360. The light also passes through lenses 1358, and may be directed to the reaction chambers through optical fiber. The reaction chamber is provided on an example microfluidic disk having a substrate layer made of quartz 1382. The reaction chamber is shown to comprise inactivated Ebola antigen 1372; a competitive antigen 1374; PEG, glycine (which prevents non-specific binding) 1376; Ebola monoclonal antibodies 1378; and a layer of protein NG (which minimizes non-specific binding and ensures proper monoclonal antibody orientation) 1380. The binding of inactivated Ebola antigen to Ebola monoclonal antibodies induces unique backscattering. Though FIG. 15 shows the light source being provided to an individual reaction chamber, in some embodiments, a central light source may be provided and waveguides may direct incident radiation to each of the reaction chambers. Optionally, as described in relation to FIG. 18, light may be provided centrally of the microfluidic disk and the light may be dispersed within the disk to the reaction chambers; backscattered radiation may thereafter be detected at the peripheries of the reaction chambers, or centrally of the disk.

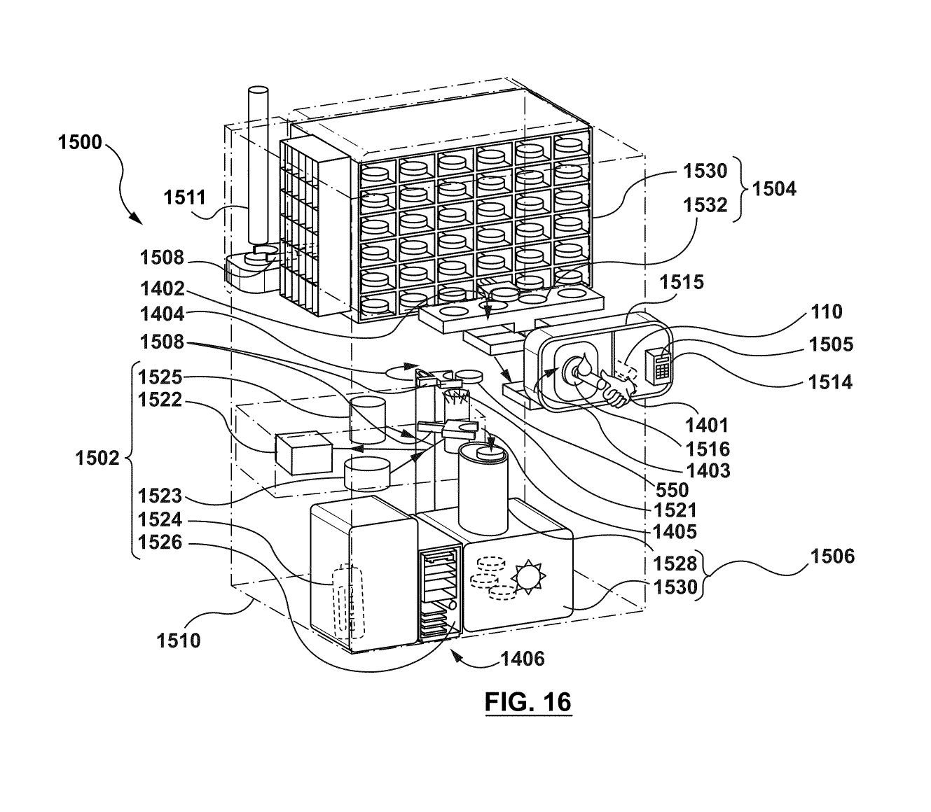

Referring now to FIG. 16, a further apparatus for pathogen detection 1500 is shown. Similarly to apparatus 1000, the apparatus 1500 comprises components for carrying out the steps of the method 200. Particularly, the apparatus 1500 comprises a detector unit 1502, a storage unit 1504 and a disposal unit 1506. The apparatus 1500 further comprises a sample handling unit 1508, a casing 1510 and associated structural components to provide the apparatus with a rigid structure, an environmental sampling unit, a user input unit (shown as fingerpad 1514), an input port 1516, and a collector storage vessel 1515 comprising collectors 110, such as individually packaged sterile swabs. The illustrated detector unit 1502 comprises a laser source 1525, a white light source 1523, a spectrometer 1522, as well as a power supply 1524, an optic fiber to light the sample, and CPU/remote signaling equipment 1526.

The storage unit 1504 provides storage and optionally refrigeration for a plurality of microfluidic disks (such as 550, 950) (at element 1530), similarly to storage unit 1004. The storage unit further comprises a port 1532 from which a microfluidic disk may be retrieved from the storage unit. The disposal unit 1506 comprises an autoclave 1530 and a used sample port for receiving a microfluidic disk 550 for disposal 1528. Though the apparatus 1500 is illustrated in relation to use with a microfluidic disk, the apparatus could be configured to receive a sample on a single assembly 300 or in a vial, with necessary modifications.

The sample handling unit 1508 (referred to as “robotics”) comprises components, such as a grabber arm, electric motors and various mechanical linkages, controlled by a robotics controller for moving microfluidic disks and other components within the apparatus 1500.

The environmental sampling unit comprises an environmental sampling port 1511, and an environmental sensor unit to detect aerosol transmissible pathogens. Upon detection of aerosol transmissible pathogens, an alarm signal may be output. The alarm signal may be further processed by the device 140 or provided over a network to remote computers 160. In some embodiments, the environmental sampling port may provide a microfluidic disk 550 in proximity to the port for exposing the disk to ambient air, the reaction of which may be detected in detection operations to determine the presence of particular pathogens in the sample which react with the ambient air.

FIG. 16 illustrates an implementation of the steps of method 200 by the apparatus 1500. At block 1401, fingerpad 1514 receives user input from a subject 1512. The user input may be received in order to automatically (or manually) select a particular type of microfluidic disk 550, configured for particular pathogens, as described in relation to FIG. 6. For example, a display 1505 of the finger pad may provide a series of questions to the subject, such as the subject's history of presenting illness, and accept answers (such as symptoms) on the fingerpad 1514. The answers may be analyzed to select an appropriate microfluidic disk 550. At block 1402, the selected microfluidic disk is retrieved from the storage unit 1504 by the robotics and may be positioned for receiving a sample from a sample collector. At block 1403, the subject receives a sample collector, such as a sterile swab. The subject may receive the collector from a storage vessel comprising individually wrapped collectors. Alternately, the collector may be dispensed from an output port of the apparatus by the robotics. The subject then deposits a sample onto the collector and inputs the collector back into the apparatus through the input port 1516. Optionally, the output port and the input port may be a shared port into the apparatus. Once received, the sample is applied to the microfluidic disk. The input port may be aligned with the saliva sample port 556 of the microfluidic disk for depositing the sample onto the microfluidic disk. If the collector is a collector 1201, a fluid connecting end may be broken off and left in the sample port 556. The microfluidic disk may then be processed. For example, the microfluidic disk may be centrifuged to spread the sample. Further, a buffer solution may be applied to the microfluidic disk. At block 1404, detection operations 220 are then performed by the detector unit 1502 for generating detection signals. For example, as shown by element 1521, light from a laser light source and a white light source may be applied (optionally through an optic fiber) to the microfluidic disk. At block 1405, the microfluidic disk 550 is provided to the disposal unit 1506 for sterilization in the autoclave 1506. At block 1406, analysis is performed on detection signals from the detection operations, as at block 230 of method 200.

In the following, machine learning implementations of the systems and methods described above will be described in additional detail.

In some embodiments described above, neural networks data analysis may be utilized for the identification of reference signals, matching of detected signals and reference signals, and otherwise detecting the presence or absence of a pathogen based on the detected signals, employing various learning techniques. These embodiments may be carried out by a processor of device 140, or by remote computers 160 in communication with device 150 over the network, optionally during the analysis stage 230. As described above, detection signals may be received from a detector 130 at the device 140.

Analysis may be implemented by providing input data to a neural network, such as a feed-forward neural network, for generating at least one output. The neural networks described below may have a plurality of processing nodes, including a multi-variable input layer having a plurality of input nodes, at least one hidden layer of nodes, and an output layer having at least one output node. During operation of a neural network, each of the nodes in the hidden layer applies a function and a weight to any input arriving at that node (from the input layer or from another layer of the hidden layer), and the node may provide an output to other nodes (of the hidden layer or to the output layer). The neural network may be configured to perform a regression analysis providing a continuous output, or a classification analysis to classify data. The neural networks may be trained using supervised or unsupervised learning techniques, as described above. According to a supervised learning technique, a training dataset is provided at the input layer in conjunction with a set of known output values at the output layer. During a training stage, the neural network may process the training dataset. It is intended that the neural network learn how to provide an output for new input data by generalizing the information it learns in the training stage from the training data. Training may be effected by backpropagating error to determine weights of the nodes of the hidden layers to minimize the error. The training dataset, and the other data described herein, can be stored in a database connected to the device 140 or otherwise accessible to device 140 or remote computers 160. Once trained, or optionally during training, test data can be provided to the neural network to provide an output. A neural network may thus cross-correlate inputs provided to the input layer in order to provide at least one output at the output layer. Preferably, the output provided by a neural network in each embodiment will be close to a desired output for a given input, such that the neural network satisfactorily processes the input data.

According to a first embodiment, a neural network interprets received detection signals from a detector. The selected neural network may be configured as a convolutional feed-forward neural network. Optionally, the neural network may receive at least one detection signal as an input and output an indication of whether a particular detection signal relates to a reference signal indicating the presence of a bound desired substance, or a reference signal indicating that a desired substance has not bound the sensing surface, as described above. Accordingly, during use at least a measured detection signal, or some scaled or otherwise modified value thereof, will be provided to the neural network as an input. Optionally, additional data may be provided to the input layer of the neural network to assist in interpreting received detection signals from a detector. Combinations of data could be provided at the input layer, including: protein interaction data (e.g. of the pathogen), and genomic/nucleic acid data of the pathogen, subject and/or desired substance (i.e. biomarker). Accordingly, high-throughput genomic sequencing of the subject/pathogen may be required, but could be performed by remote computers 160 and need not be carried out at the local device 140. Further input data could include mass spectrometry data (e.g. from pathogen protein sequencing). Still further data inputs may include, time series genomic data of various pathogens and protein interaction in the pathogen and host, subject history (e.g. flight history or medical history). This embodiment may thus cross-correlate various inputs to provide an output to aid in interpreting a detection signal to determine whether a pathogen has been detected. The additional data may be received from a third-party data repository.

An output indicative of detection of a pathogen may result in notification being generated to alert a local medical professional; alert a medical professional already associated with the patient; alert an expert in the healthcare field with special knowledge of the specimen; and, alerting local health or police authorities as required by law for diagnosis of health conditions. Further, an output indicative of detection of a pathogen may result in generating a request for human ground-truthing of the detection signal/sample. For example, a microscopic image of a sample can be electronically transmitted to ground truther for assessment. Further, the patient may be advised of any immediate actions that they should take for their own immediate health and safety and for the public in the vicinity.

In another embodiment, a neural network is applied to compensate for nanoscale and quantum realm detection limitations. Particularly, a detection signal is provided to a neural network, with a desired output compensating for defects in the detection signal that may be caused by limitations of imaging in the nano-realm. The input layer may receive data relating to the detection modality and an input detection signal for detection of viruses, bacteria, fungi, parasites, human host (i.e. subject) cells, disease biomarkers. The neural network may be trained such that the output layer provides a clean detection signal compensating for signal defects. Particularly, the neural network may be trained with a training dataset comprising, at the input layer, detection signals comprising nano-realm defects, and with associated clean detection signals at the output layer for viruses, bacteria, fungi, parasites, human host cells, disease biomarkers for detection modalities. The output of the trained neural network may provide a processed detection signal similar to known reference signals for particular detection modalities such that processing by the neural network remedies some defects and limitations of received detection signals.

In another embodiment, a neural network is applied to drive evolution of the choice of amplification substance provided to each assembly (and/or reaction chamber 500 of the microfluidic disks 550). Particularly, selection of amplification substance may compensate for mutation, pleomorphism and polymorphism of pathogens to ensure that appropriate amplification substances are selected to maximize likelihood of detecting a pathogen. Accordingly, inputs including a combination of time series genomic data of the pathogen and/or human host cells, and data relating to a plurality of desired substances (e.g. disease biomarkers) may be provided to a neural network trained to provide an output indicating which amplification substance(s) should be selected. A sensing surface comprising, for example, a selected biosensor immunoassay antigen capture mechanism could then be provided to each reaction chamber 500 of a microfluidic disk 550. The embodiment may similarly require high-throughput genomic sequencing of the subject/pathogen, as well as mass spectroscopy data.

In another embodiment, a neural network based predictive output machine is provided. Particularly, the machine learning predictive output machine may receive inputs comprising time series genomic data of a subject in order to provide an output indicative of a clinical outcome. To provide time series inputs, samples may be taken and sequenced from a subject and/or pathogen over a period of time to maintain or improve the accuracy of the neural network over time. To train the neural network a training dataset may comprise known inputs of the specified data types as well known associated clinical outcomes. Further data inputs may include, time series genomic data of various pathogens and protein interaction in the pathogen and host, subject history (e.g. flight history or medical history), subject condition (e.g. resistivity to aids).

Although the invention has been described with reference to certain specific embodiments, various modifications thereof will be apparent to those skilled in the art. The scope of the claims should not be limited by the preferred embodiments, but should be given the broadest interpretation consistent with the description as a whole.

We claim:

1. A system for detecting a pathogen comprising:
a. a collector for collecting a sample from a subject;
b. an assembly for receiving the sample, the assembly comprising:
i. a substrate layer; and
ii. an amplification layer comprising at least one amplification substance immobilized and functionalized to the substrate layer for interacting with a desired substance associated with the presence of the pathogen in the sample;
c. a detector for receiving the assembly and for generating detection signals from the received assembly according to at least one detection modality; and
d. a computing device for analyzing the detection signals and for determining presence or absence of the pathogen in the sample.

2. The system of claim 1, wherein the amplification layer comprises a plurality of amplification substances arrayed on the substrate layer for interacting with a plurality of desired substances associated with the presence of the pathogen.

3. The system of claim 1, wherein analyzing the detection signals comprises determining whether the detection signals relate to first reference signals indicating the absence of the pathogen, or second reference signals indicating the presence of the pathogen.

4. The system of claim 3, wherein the computing device comprises a neural network for receiving the detection signals at an input layer and for generating the determination at an output layer.

5. The system of claim 4, wherein the neural network receives additional data at the input layer relating to any one of the subject, the pathogen, or the subject and the pathogen for generating the determination.

6. The system of claim 5, wherein the additional data is flight history of the subject.

7. The system of claim 1, wherein the substrate layer comprises a passive layer made of silica and an active layer made of a metal.

8. The system of claim 1, wherein the system comprises a buffer application unit for discharging a buffer solution onto the assembly for flushing away undesired substances from the assembly.

9. The system of claim 1, wherein the detector comprises a tunable radiation source for emitting a plurality of discrete wavelengths of radiation to the assembly for generating detection signals.

10. The system of claim 1, wherein the at least one detection modality comprises backscattering interferometry of the substrate layer.

11. A method for detecting a pathogen comprising:
a. receiving a sample from a subject using a collector;
b. providing the sample to an assembly, the assembly comprising:
i. a substrate layer; and
ii. an amplification layer comprising at least one amplification substance immobilized and functionalized to the substrate layer for interacting with a desired substance associated with the presence of the pathogen in the sample;
c. providing the assembly to a detector, the detector configured to generate detection signals corresponding to the assembly according to at least one detection modality; and
d. initiating the determination, by a computing device having a processor, of the presence or absence of the pathogen in the sample by analyzing the detection signals.

12. The method of claim 11, wherein the amplification layer comprises a plurality of amplification substances arrayed on the substrate layer for interacting with a plurality of desired substances associated with the presence of the pathogen.

13. The method of claim 11, wherein analyzing the detection signals comprises determining whether the detection signals relate to first reference signals indicating the absence of the pathogen, or second reference signals indicating the presence of the pathogen.

14. The method of claim 13, wherein the computing device comprises a neural network for receiving the detection signals at an input layer and for generating the determination at an output layer.

15. The method of claim 14, wherein the neural network receives additional data at the input layer relating to any one of the subject, the pathogen, or the subject and the pathogen for generating the determination.

16. The method of claim 15, wherein the additional data is flight history of the subject.

17. The method of claim 11, wherein the substrate layer comprises a passive layer made of silica and an active layer made of a metal.

18. The method of claim 11, further comprising discharging a buffer solution onto the assembly by a buffer application unit for flushing away undesired substances from the assembly.

19. The method of claim 11, wherein the detector comprises a tunable radiation source for emitting a plurality of discrete wavelengths of radiation to the assembly for generating detection signals.

20. The method of claim 11, wherein the at least one detection modality comprises backscattering interferometry of the substrate layer.
